SEQUENCE LISTING (22 sequences)

SEQ ID NO: 1 - protein, 866 aa, Homo sapiens
MEEGNNNEEVIHLNNFHCHRGQEWINLRDGPITISDSSDEERIPMLVTPAPQQHEEEDLDDDVILTEDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRF

SEQ ID NO: 2 - protein, 1049 aa, Homo sapiens
MACIHYLRENREKFEAFIEGSFEEYLKRLENPQEWVGQVEISALSLMYRKDFIIYREPNVSPSQVTENNFPEKVLLCFSNGNHYDIVYPIKYKESSAMCQSLLYELLYEKVFKTDVSKIVMELDTLEVADEDNSEISDSEDDSCKSKTAAAAADVNGFKPLSGNEQLKNNGNSTSLPLSRKVLKSLNPAVYRNVEYEIWLKSKQAQQKRDYSIAAGLQYEVGDKCQVRLDHNGKFLNADVQGIHSENGPVLVEELGKKHTSKNLKAPPPESWNTVSGKKMKKPSTSGQNFHSDVDYRGPKNPSKPIKAPSALPPRLQHPSGVRQHAFSSHSSGSQSQKFSSEHKNLSRTPSQIIRKPDRERVEDFDHTSRESNYFGLSPEERREKQAIEESRLLYEIQNRDEQAFPALSSSSVNQSASQSSNPCVQRKSSHVGDRKGSRRRMDTEERKDKDSIHGHSQLDKRPEPSTLENITDDKYATVSSPSKSKKLECPSPAEQKPAEHVSLSNPAPLLVSPEVHLTPAVPSLPATVPAWPSEPTTFGPTGVPAPIPVLSVTQTLTTGPDSAVSQAHLTPSPVPVSIQAVNQPLMPLPQTLSLYQDPLYPGFPCNEKGDRAIVPPYSLCQTGEDLPKDKNILRFFFNLGVKAYSCPMWAPHSYLYPLHQAYLAACRMYPKVPVPVYPHNPWFQEAPAAQNESDCTCTDAHFPMQTEASVNGQMPQPEIGPPTFSSPLVIPPSQVSESHGQLSYQADLESETPGQLLHADYEESLSGKNMFPQPSFGPNPFLGPVPIAPPFFPHVWYGYPFQGFIENPVMRQNIVLPSDEKGELDLSLENLDLSKDCGSVSTVDEFPEARGEHVHSLPEASVSSKPDEGRTEQSSQTRKADTALASIPPVAEGKAHPPTQILNRERETVPVELEPKRTIQSLKEKTEKVKDPKTAADVVSPGANSVDSRVQRPKEESSEDENEVSNILRSGRSKQFYNQTYGSRKYKSDWGYSGRGGYQHVRSEESWKGQPSRSRDEGYQYHRNVRGRPFRGDRRRSGMGDGHRGQHT

SEQ ID NO: 3 - DNA, 5696 bp, Homo sapiens
agtgcctctatggttcttcctgtccttagtcgggagggggcgctgtgcgcaggcgcgaggtgacgccgcggcagggccggaaggagtgcagcggctgccacggagctcgcagctgcggcttctgaggagtaagcggcgcggtagcgaaggccgccgaacctgcctggctagccggcgacttgagtgacgactcttttgaaacagatggtcaccatgtttagatattagcagtcccatatatgcatgtctgcatttgaaaatggaagagggaaacaacaatgaagaggtaattcacttgaacaactttcactgccatcggggacaagagtggatcaatctccgagatgggcccatcaccatatctgactcctcagatgaggaaaggattccaatgctggtcaccccagctcctcagcagcatgaagaagaggacctggatgatgatgtcatcctgacagaagatgattctgaggatgactacggtgaatttctggatcttgggcctcctggaatctctgaattcactaagccaagtggccaaacagaaagagaacccaagcctggaccgagtcataaccaagcagcaaatgacattgtcaaccccagatcagagcagaaagtcatcatcttggaagaaggtagccttctttacacagaaagcgatcctttggaaactcagaaccagtcatccgaagactcagagacagagctgttatcaaatctaggagagtcagctgctctagcagatgatcaggccatcgaagaagactgctggttagatcatccttacttccagtctctgaaccaacagccccgtgaaataacaaaccaggtcgttcctcaggaacggcagcctgaagcagaactgggccgcttgttgtttcagcatgaattcccagggcccgcttttccaaggccggaaccccagcaaggtgggatttcaggcccctcttctcctcagcctgcccatcctctaggagagtttgaagaccagcagttagcaagtgatgatgaagagccaggtccagcctttccaatgcaagaatctcaagagcccaatttggaaaacatttgggggcaagaagctgcagaggtagatcaagagctcgttgaactactagtgaaagaaacggaagcaagatttccagatgtagcaaatgggtttattgaggaaataattcattttaagaattattatgatctgaatgtactttgtaattttcttctggaaaacccagattatccaaagagagaagacagaatcattataaatcccagtagcagtctgctggccagccaagatgagacaaagttgcctaaaatagacttttttgactattctaaattgacccctcttgaccagcgctgcttcatccaagctgctgacctcctcatggccgacttcaaagtgctcagtagtcaggacatcaagtgggccctgcacgagctcaaaggacactatgcaatcacccgaaaggccttgtctgatgccattaaaaaatggcaggagctgtcaccagaaaccagtggaaaaaggaagaagagaaaacaaatgaaccagtattcttacattgatttcaagtttgaacaaggtgacataaaaatagaaaagaggatgttctttcttgaaaataagcgacgacattgtaggtcctatgaccgacgtgctctccttccagctgtgcaacaagagcaggagttctatgagcagaaaatcaaagagatggcagagcatgaagactttttgcttgccctacagatgaatgaagaacagtatcaaaaggatggccagctgattgagtgtcgctgctgctatggggaatttccattcgaggagctgacgcagtgcgcagatgctcacttgttctgcaaagagtgtctcatcagatatgcccaagaggcagtctttggatctggaaagttggagctcagctgcatggaaggcagctgcacgtgttcgttcccaaccagtgagctggagaaggtgctcccccagaccatcctgtataagtactatgagcgaaaagccgaggaggaggttgcggcagcctacgccgacgagcttgtcaggtgcccgtcctgtagctttccggctctgttggacagtgatgtgaagaggttcagctgtcctaatcctcactgccgaaaggaaacctgtaggaagtgtcagggactctggaaagaacataatggcctcacctgtgaagagctggctgaaaaagacgacatcaagtaccgtacctctattgaagaaaaaatgactgctgcccgcattagaaaatgccacaagtgtgggactggcctcatcaaatctgaaggctgcaaccgcatgtcttgccgctgtggtgcccagatgtgctacctctgtcgagtttctattaatggatatgaccatttctgccaacatccccgctcaccaggagccccttgccaggagtgttcaagatgctctctctggaccgatcccactgaagatgatgagaagcttattgaggaaatccagaaggaggctgaagaggaacagaaaagaaagaatggagagaacaccttcaaacgcattggacccccgctggagaagcctgtggagaaggtgcagagggtggaggccctcccgaggcccgttccgcagaacctgccacagccacagatgccaccctatgccttcgcgcacccacccttccccctgcctcccgtgcggcctgtgttcaacaacttcccactcaacatggggcctatcccagccccgtacgtgccccctctgcccaacgtgcgggtcaactatgacttcggtcccatccacatgcccctggagcacaacctgcccatgcactttggcccccagccgcggcatcgcttctgatggccccgaatccccattgagcagcacaaagcccgtttggggtaggagtgtggatggagaaccctcccccaaggctggtgtctgtaccattgcatcctaagtcagcttgaagggtaggctggttttcttcccacccctttcctagaagggctactgctcctggaagagtggacggatccataataaagacgtcccaaatggtggagttcggagagagctgcgatgtgaactgcccctcccctcgcatcccccaggccaccaacggcagtccttctgccttgtccatggcataggccatagaccaggtccctgctgctcacacctgggcctctcctcggagccgacccctgggtagcaaggcagccgagagcatctccctggaggggcccacggttgggccaagggcagagggggctgcacctgcgggcctgggaagcattgctcagggtggggggctgggaccatggcccgcagaggcactgccacagctgtgagggccaagatgctgtccccccatccaaaacccgtgcgccactgcagtgagtgttgagggcacctctcctcccctcttacacctactcagatgaggcagcagcagacccatctcgcggcgggggttttgttctgttgccgcctaactttctcatcctcggtctctggaaagtcaggctgagaaatcctttcccaggccaggccgctgcggtacactggatggttctgaagctggcccattgaaagagcctcttaaggcagctgggacagaggcctggtggccctgctgggcagcccaactgctgggggagacgtttctgccaccctgggtgatgagcagcttttcccccctggctttctgggggaggagtgggcctccttagggagacaggtgaccctgggtgccacccctgccccgtgtgtgccccgggtgttctcagtggttgctgaaggcaggtagagggtgctgtccagtatcccccatgtgaaggtcacttcccttctcatggagtcagctgagcatcagctcagccctgccatgtccccactcaccctcctcgcctcctgtccggccctgggtttctagcggtgcctgaggcatcactctggcccattgacagatgagaggtctgaagccttcctggccacaggcatcactttctcctcctcctcatgccctgccttgtccttgtcgtgttgccatggggttctgagaggctgggagttcacagacctcagacacagctgagtccgacaaccattggggtggggctgcatcagtctccggagtggcccgccacctcctgaagcagggcctggcccacccaaggtgcctggggcaggcgggcaccgtcattcgctgccattggcttctcagatgtatttcaaggactaaagtgggctctaagatctaagatggcccggcgcggtggctcccgcctgtaatcccagcactttgggaggccgaggcgggcggatgagttgaggtcgggagtttgagtccccgtctctactaaaaatacaaaattagccggacaaggtggcgcatgcctataatcccaggtactcaggaggctgaggcaggagaatcacttgaacctgggaggcagaggttgcagtgagccaagattgtgccactgcactccagcctgagcaacaaaagcaaaactctatctttaaaaaaaaaaaaaaaaaaactaagatacccattctttctgcctttgcccaggatggaggctttgaagacaggtggtcacctggacctggggtaagggtgcgcagggaggccgggcttgggggcagcctgctgctatgtccttgctggcgcccatacctgttggactgcctgaactttgctactagcctgaacgacagtgccttggaaaggcaggctccatgcggcagcaacaggattgtcttcggtaccttaaatatgctccctgtgcttccccagggaggatctctgggagcagagggcggctgtagacatcacagctgaagaggtgacctcaggccctgggagcctcagctccctctgccctgtaccctaggcacgtggctccagcaagagccccaggcaggacaaagggagcaggcagcagctgcagaaacagccctagacaggtgctggcctcaaccctggcaggggagggggagcccagagtgatgtttctcctaaggccaaaccccaggggtgggtcatttgagaggagttccagctgttctggccacactgggctggtggggaaggagcggcggcctctttccttggagggtccctgattcctgaggatcctccccgagccaggcacgggggaccacagggctgccgtgagcacccaccctggttctccaggcccctctgtccagaggcagggcctgaacctcgggctcctgcctggaccagtgacagcattggggagagaagcactgggggtcttcagaccagccagcaaggggccgtgccagtgcccagagagcaggtcagacaacaggcaaccctgaggcagctggctccctctctctgccctgcagggcttgccgcagggcgtgaggctccccagccacagccccctcttacttgccttaaaactggagagaggaatctagtatttgcacttagcaaaatatttaaaaactgaaaaaaatgtgcatgatctgtcactttgtataaaaacaagaaacgattagttcattaaactttttctttaccttctggtagtaaaaatagataaaataattccgtttcaactactgtgtgtaaaaagcctgtatcatatgtaactgggacttttgtaaataaatgtttgtgcgctctgctccctccaaaaaaaaaaaaaaaaaa

SEQ ID NO: 4 - DNA, 7068 bp, Homo sapiens
cccattgtaacgccggagtgggacgctgctttgcgtctgaaaggtgcaaattgaaactcgaaagcccgcaaattaacggggtgtctctctcctgatgaagaggtattgcactctcagtctcgccatgttgaagtcagaatggcctgtattcactatcttcgagagaacagagagaaatttgaagcgtttatagaaggatcatttgaagaatatttaaagcgtttggaaaatccacaggaatgggtaggacaagtggaaataagtgccctttctcttatgtacaggaaagattttataatttatcgggaaccaaatgtttctccttcacaagtaacagaaaataattttcctgaaaaggtgttactgtgtttttcaaatggaaatcattatgatattgtgtatcccataaagtataaagaaagctctgctatgtgtcagtctctcctttatgaattgctgtatgagaaggtatttaaaactgatgttagtaaaattgtgatggaactagacacgttggaagtagctgatgaagataacagtgaaatatcagattcagaggatgacagttgcaagagtaagactgctgctgctgctgctgatgtgaatggatttaaacctttgtcaggcaatgagcagctgaagaacaatgggaactctactagcctgcctttgtctagaaaggttcttaagtcactcaatcctgcagtctatagaaatgtggaatatgaaatttggctgaagtctaaacaagctcagcaaaaacgtgattattccattgctgctggcttacaatatgaagttggagacaaatgtcaagttaggttggatcacaatggaaaatttttgaatgcagatgttcaaggaattcattctgagaatggaccagttttggttgaagaactgggaaagaagcacacatcaaagaacctcaaggcacctcccccagaaagctggaacacagtgtcagggaagaagatgaaaaaaccttccacttctggacaaaatttccattctgatgtggattacagagggccaaagaatccaagcaagccaataaaagccccatcagcactacctcctcgactgcagcatccttcaggagtaagacaacatgcgttctctagtcattcttcagggtcacagtctcagaaattctccagtgagcacaaaaatcttagccggacaccttcacagatcataagaaaacctgatcgtgaaagagttgaggattttgatcacacaagtcgagaatctaactatttcggcctttccccagaagagcgcagagagaagcaagctatagaagaatcccgtttactctatgagattcagaacagagatgaacaggctttcccagccctttccagctcatcagtcaatcagtcagcttctcagagtagcaatccatgtgtccagagaaaatcatcacatgtaggtgatagaaaaggaagcaggcggagaatggatacagaagaacgaaaagacaaagactctattcatggacatagtcagttggataaaagacccgaaccaagcacattggagaatattactgatgataaatatgcaacagtttcatcaccatcaaagtcaaagaagttagagtgcccttctcctgcggaacaaaagccagcagaacatgtgtctttgtcaaatccagctccccttctagtttctccagaggtacatctaactcctgcggtgccttctttaccagccactgtgccagcctggccaagtgaacctacaacttttggaccaacaggtgtccctgctccaattcccgttttgtcagtgacacagactttgaccactggacctgattcagctgtatcccaagctcatttaacaccctctccagttcctgtgtcaatacaggcagttaaccagcccttgatgcctttgcctcagacattgagcctttatcaagacccactctatcctgggtttccttgtaatgaaaagggagatcgagccattgtaccaccttattcactgtgtcagactggggaggacctacctaaagataagaatattcttcgattcttcttcaatcttggtgtgaaggcatacagttgtcctatgtgggccccacattcttacctgtaccctctgcaccaggcctacctggcagcctgcaggatgtacccaaaggtccctgtccctgtttatcctcataatccctggttccaagaggctcctgctgctcagaatgaaagtgattgtacctgtactgatgcccactttcctatgcagactgaggccagtgttaatggtcaaatgccacagccagagattggaccgccgacattttcttcacctctggttatccctccatctcaggtgtctgaaagtcatggacaattgtcttaccaggctgatcttgaatctgagacccctgggcagcttctgcatgctgattatgaagagtcactaagtggcaagaatatgttcccccagccatcttttggacccaatccattcttaggcccagttcctattgcacctcctttctttcctcatgtttggtatgggtacccttttcagggattcatagaaaatccagtaatgaggcagaatattgtcctgccctctgatgaaaaaggagaattggatctgtctctggaaaatctggatctgtctaaagattgtggttcagtttcaacagtagatgagtttccagaagccaggggtgaacatgtacattctctccctgaagcaagtgtgagcagtaagccggacgaaggccggacagagcaatcttcccagacacgaaaggcagatacggcattggcttccatccctcctgtagcagagggaaaggctcatcctcccactcagattctaaacagagagagagaaactgtgcctgttgaacttgaacctaaaaggaccattcaaagcctgaaagaaaaaacagaaaaagtaaaagatcctaagactgctgctgatgtggtcagccctggggccaactctgttgatagcagagtgcaaagaccaaaagaagagagttcagaagatgaaaatgaagtgtctaatattttgagaagtggtagatccaagcagttctataatcaaacttatggaagcaggaagtacaaaagtgattggggctattctggtaggggtggatatcaacatgtgagaagtgaggagtcctggaaaggacagccaagcagaagtcgggatgaaggttatcagtaccatcgaaatgtcagagggcgaccatttaggggagataggaggagatcagggatgggagatggccataggggacagcacacttgatggttgttgccgaagtattttctaacagaaactcttaggtggaatgtttctgaaggctttaaaaaaaatacaataaagtaaaaaccgcagttggaactctttcctctccccagccccccttaccttcactcccatactggacaagacattttggacatgagttcaagggggcaggttaattcctggcattgccagttttcttcattcagaatctgtttgttgatcaagtgactgtccccgtaaaaagtagaagataagtttgttaaagtattttttataaacagcttaatataaaacattatagatttagtgtttggcttttttccttataagtttataagtttgatttcagatgttggaaatgtgtgctttatagtgtttactaaagtgacaagaaaatataccatttgacacactatagattccaaacataacgttgcaccaaatagaaatgtatattttattatgcaatgccttagtcataaactgggctcaaacaattctcagcctaaaacactgttgtcttttaaaatgcttccaacccaaaggcctttcatctcaatatctgtcaaacttgaataatgtcttctctttaatattcacataaggcagcctattaactgtgtcttagcaatgttgtatatagttcttttaatattcataaggtatatttttgaattttggtatttgttgagattgcatagaagaatagtaggaaatccaatgaaatggctgtttccaccaagaattcttagcactggtgtaaatatcatggtgcctactcgaaagaaatgtagatgctatgcattttgaaaataatctgcatcagttagaactgcagaagtttttttaatgccactttaacactaatgcactgacagattctaaatattttgtgagaagaaatgttaaaaatttttagcttgacaggtttctagagttgttttcttcctcccccccccccccgcaccattggttgtgtatcactttcactttacacttgcatgttctacttgtcagggctggaactattgtatagaagataatatgattgtgacttctagttcttttctagaggatgctgctagaaaatggttttgctttgtattttatagcttgttacccttaggtaggtaacatcttacattctgttttcttccagtctttgcagtattcactagaggttccctttgcatgttgcactctgttctcatttggagattgaactctgaattaacacagtattcattcatctgtgttattttgtaaaaataactgtagcttatattctttatttgtgcataccaatgtgaaaatccactcttttattatgttgagattatcttctgatcaggaagtttgagtgctatgcaaataacatagtagtaatttctctttgcatgccatgtgtaatatacctagccaaaaccagatgcggtgactttctttttttgtaaagcaaattacttaagaaaatacaattcacttgaatttgacaaactgaaacagatgggttttctgggttctctgataacctatgggaatgtttggatgccagttgggctagtgaatcgccactgtactgtaataatggttatttacctctgtgctgttttaattacctaacgtctgtgtaactgctgatttgagtagtgattagcatttagataatgtaacaggattttagagagcactttgaaagataacattttattatgatggtgctaggtaaaaaattgtaaagactgggatggttgtgtggaaaacttttcttgagagaacctattgaaaggaatttgttgtcaccttcttaactagaatagttgttgaaggtaccgcttaagccatttttttcttaacatcggtggctcagttaaggaatgagaagatagagggtcctccactttcagctcatgcttttttatgtaggactagcttacctgattttaatgaaaatcacattttgatacaaacaccagatttgaaagttcaatgtgtttgatctcaggagtccaaaataagttgttgcttctagtgtcctgcattttattgactggggtttgatctcagcttctgaaaatgtttaaaaattggaaggaaatactactcatatcttcattgtcactttgtcttacctcaattgagtaacagaatatagctcctgaaggaaacactgatttgctttctgtttcccatatctcacctctaggatatgatggaagaagtgcttgaaagagaattttagcagatggtacttttctcagtgacctctggaataaaagaactgacttttgatagttttaaaattaatctttgcatgaaggttggtttcgctttttttcctttaagtttgtttttaaatgtattagtccaggggattttgccttatgctgaggtcaaactaaggataagcaagcttttgtccttcattttaactgttatgtcatactgttatgttgacatatttctttataagagaatagaggcaaaagtatagaactgaggatcatttgtatttttgagttggaaattatgaaacttcaccatattatgatcatacatattttgaagaacagactgaccaaagctcacctgttttttgtgttaggtgctttggctgaacttgattccagcccccttttccctttggtgttgtgtatgtctcttcatttcctctcaaatcttcaactcttgccccatgtctccttggcagcaggatgctggcatctgtgtagtcctcatactgtttactgataacccacaaattcattttcatggcagacctaagctcagaccctgccttgtcctggcaagcataggttttcccagaccacaatgctgatatatcttatttaatgcagactgtattcttgcagcgtgttgtttcctttgtgactcaaggttgaattaaaatgcctgtgaactgcaaacttgcttatgattgacttggtgcaataaagttcctttctaaccatttctggtttagaaaacattcagccatcctagaaactagcaatatttatttttgcctctttcgttttaccttcatttttagatgaggtaaatgttttgtcttttacttaagggtactaagcaaggtccactctagtgtagctgaagagaatgcatatttggctacactatttctgtttactgtattttgcttcctactttatgtctaaatgctattgtttgtttaaaatcctaataatgtctcatttccaatatgttattttcagataactaaggttttgctactaatgtcagccatttactctcccactaattgggattattttttaaaagaggtctttctccccgaaatacaagttttagaaaatcttagaagaggggtgggtgcatgtcttaatgctgggaagcggcaactttggggttgaacttacttgaatggattaaaaattgcattgtgttacagagctagaaaattttatgtatgaaaaacgataatagccttacactgagtacaaataatactttaagagcatgtgaagatgtgatcatttgtgatgttacttgtaaaaaaaagtatatatacatatcttttgaagtgttaaaaggaaactagtactttatattctttatcatatcactttttgtaagtgttgaatatattcctgtttatttttctatgttctgttttgtagccttaagaagtgtttcaaacatatctgaatgtataaaataagagtaaatgccctacatggtgtgatgctgcattatatataaaactgtgtgcatatattaaattttgtctcttga

SEQ ID NO: 5 - protein, 923 aa, Homo sapiens
MEEGNNNEEVIHLNNFHCHRGQEWINLRDGPITISDSSDEERIPMLVTPAPQQHEEEDLDDDVILTETNKPQRSRPNLIKPAAQWQDLKRLGEERPKKSRAAFESDKSSYFSVCNNPLFDSGAQDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRF

SEQ ID NO: 6 - DNA, 5867 bp, Homo sapiens
agtgcctctatggttcttcctgtccttagtcgggagggggcgctgtgcgcaggcgcgaggtgacgccgcggcagggccggaaggagtgcagcggctgccacggagctcgcagctgcggcttctgaggagtaagcggcgcggtagcgaaggccgccgaacctgcctggctagccggcgacttgagtgacgactcttttgaaacagatggtcaccatgtttagatattagcagtcccatatatgcatgtctgcatttgaaaatggaagagggaaacaacaatgaagaggtaattcacttgaacaactttcactgccatcggggacaagagtggatcaatctccgagatgggcccatcaccatatctgactcctcagatgaggaaaggattccaatgctggtcaccccagctcctcagcagcatgaagaagaggacctggatgatgatgtcatcctgacagaaacaaataaacctcagagatcacgacccaatctcatcaaaccagctgcccagtggcaagatctgaaaaggttgggagaagaaaggcctaaaaagtctagagcagcatttgaatcagataagagcagctatttttcagtgtgtaacaacccattgtttgattctggggcacaggatgattctgaggatgactacggtgaatttctggatcttgggcctcctggaatctctgaattcactaagccaagtggccaaacagaaagagaacccaagcctggaccgagtcataaccaagcagcaaatgacattgtcaaccccagatcagagcagaaagtcatcatcttggaagaaggtagccttctttacacagaaagcgatcctttggaaactcagaaccagtcatccgaagactcagagacagagctgttatcaaatctaggagagtcagctgctctagcagatgatcaggccatcgaagaagactgctggttagatcatccttacttccagtctctgaaccaacagccccgtgaaataacaaaccaggtcgttcctcaggaacggcagcctgaagcagaactgggccgcttgttgtttcagcatgaattcccagggcccgcttttccaaggccggaaccccagcaaggtgggatttcaggcccctcttctcctcagcctgcccatcctctaggagagtttgaagaccagcagttagcaagtgatgatgaagagccaggtccagcctttccaatgcaagaatctcaagagcccaatttggaaaacatttgggggcaagaagctgcagaggtagatcaagagctcgttgaactactagtgaaagaaacggaagcaagatttccagatgtagcaaatgggtttattgaggaaataattcattttaagaattattatgatctgaatgtactttgtaattttcttctggaaaacccagattatccaaagagagaagacagaatcattataaatcccagtagcagtctgctggccagccaagatgagacaaagttgcctaaaatagacttttttgactattctaaattgacccctcttgaccagcgctgcttcatccaagctgctgacctcctcatggccgacttcaaagtgctcagtagtcaggacatcaagtgggccctgcacgagctcaaaggacactatgcaatcacccgaaaggccttgtctgatgccattaaaaaatggcaggagctgtcaccagaaaccagtggaaaaaggaagaagagaaaacaaatgaaccagtattcttacattgatttcaagtttgaacaaggtgacataaaaatagaaaagaggatgttctttcttgaaaataagcgacgacattgtaggtcctatgaccgacgtgctctccttccagctgtgcaacaagagcaggagttctatgagcagaaaatcaaagagatggcagagcatgaagactttttgcttgccctacagatgaatgaagaacagtatcaaaaggatggccagctgattgagtgtcgctgctgctatggggaatttccattcgaggagctgacgcagtgcgcagatgctcacttgttctgcaaagagtgtctcatcagatatgcccaagaggcagtctttggatctggaaagttggagctcagctgcatggaaggcagctgcacgtgttcgttcccaaccagtgagctggagaaggtgctcccccagaccatcctgtataagtactatgagcgaaaagccgaggaggaggttgcggcagcctacgccgacgagcttgtcaggtgcccgtcctgtagctttccggctctgttggacagtgatgtgaagaggttcagctgtcctaatcctcactgccgaaaggaaacctgtaggaagtgtcagggactctggaaagaacataatggcctcacctgtgaagagctggctgaaaaagacgacatcaagtaccgtacctctattgaagaaaaaatgactgctgcccgcattagaaaatgccacaagtgtgggactggcctcatcaaatctgaaggctgcaaccgcatgtcttgccgctgtggtgcccagatgtgctacctctgtcgagtttctattaatggatatgaccatttctgccaacatccccgctcaccaggagccccttgccaggagtgttcaagatgctctctctggaccgatcccactgaagatgatgagaagcttattgaggaaatccagaaggaggctgaagaggaacagaaaagaaagaatggagagaacaccttcaaacgcattggacccccgctggagaagcctgtggagaaggtgcagagggtggaggccctcccgaggcccgttccgcagaacctgccacagccacagatgccaccctatgccttcgcgcacccacccttccccctgcctcccgtgcggcctgtgttcaacaacttcccactcaacatggggcctatcccagccccgtacgtgccccctctgcccaacgtgcgggtcaactatgacttcggtcccatccacatgcccctggagcacaacctgcccatgcactttggcccccagccgcggcatcgcttctgatggccccgaatccccattgagcagcacaaagcccgtttggggtaggagtgtggatggagaaccctcccccaaggctggtgtctgtaccattgcatcctaagtcagcttgaagggtaggctggttttcttcccacccctttcctagaagggctactgctcctggaagagtggacggatccataataaagacgtcccaaatggtggagttcggagagagctgcgatgtgaactgcccctcccctcgcatcccccaggccaccaacggcagtccttctgccttgtccatggcataggccatagaccaggtccctgctgctcacacctgggcctctcctcggagccgacccctgggtagcaaggcagccgagagcatctccctggaggggcccacggttgggccaagggcagagggggctgcacctgcgggcctgggaagcattgctcagggtggggggctgggaccatggcccgcagaggcactgccacagctgtgagggccaagatgctgtccccccatccaaaacccgtgcgccactgcagtgagtgttgagggcacctctcctcccctcttacacctactcagatgaggcagcagcagacccatctcgcggcgggggttttgttctgttgccgcctaactttctcatcctcggtctctggaaagtcaggctgagaaatcctttcccaggccaggccgctgcggtacactggatggttctgaagctggcccattgaaagagcctcttaaggcagctgggacagaggcctggtggccctgctgggcagcccaactgctgggggagacgtttctgccaccctgggtgatgagcagcttttcccccctggctttctgggggaggagtgggcctccttagggagacaggtgaccctgggtgccacccctgccccgtgtgtgccccgggtgttctcagtggttgctgaaggcaggtagagggtgctgtccagtatcccccatgtgaaggtcacttcccttctcatggagtcagctgagcatcagctcagccctgccatgtccccactcaccctcctcgcctcctgtccggccctgggtttctagcggtgcctgaggcatcactctggcccattgacagatgagaggtctgaagccttcctggccacaggcatcactttctcctcctcctcatgccctgccttgtccttgtcgtgttgccatggggttctgagaggctgggagttcacagacctcagacacagctgagtccgacaaccattggggtggggctgcatcagtctccggagtggcccgccacctcctgaagcagggcctggcccacccaaggtgcctggggcaggcgggcaccgtcattcgctgccattggcttctcagatgtatttcaaggactaaagtgggctctaagatctaagatggcccggcgcggtggctcccgcctgtaatcccagcactttgggaggccgaggcgggcggatgagttgaggtcgggagtttgagtccccgtctctactaaaaatacaaaattagccggacaaggtggcgcatgcctataatcccaggtactcaggaggctgaggcaggagaatcacttgaacctgggaggcagaggttgcagtgagccaagattgtgccactgcactccagcctgagcaacaaaagcaaaactctatctttaaaaaaaaaaaaaaaaaaactaagatacccattctttctgcctttgcccaggatggaggctttgaagacaggtggtcacctggacctggggtaagggtgcgcagggaggccgggcttgggggcagcctgctgctatgtccttgctggcgcccatacctgttggactgcctgaactttgctactagcctgaacgacagtgccttggaaaggcaggctccatgcggcagcaacaggattgtcttcggtaccttaaatatgctccctgtgcttccccagggaggatctctgggagcagagggcggctgtagacatcacagctgaagaggtgacctcaggccctgggagcctcagctccctctgccctgtaccctaggcacgtggctccagcaagagccccaggcaggacaaagggagcaggcagcagctgcagaaacagccctagacaggtgctggcctcaaccctggcaggggagggggagcccagagtgatgtttctcctaaggccaaaccccaggggtgggtcatttgagaggagttccagctgttctggccacactgggctggtggggaaggagcggcggcctctttccttggagggtccctgattcctgaggatcctccccgagccaggcacgggggaccacagggctgccgtgagcacccaccctggttctccaggcccctctgtccagaggcagggcctgaacctcgggctcctgcctggaccagtgacagcattggggagagaagcactgggggtcttcagaccagccagcaaggggccgtgccagtgcccagagagcaggtcagacaacaggcaaccctgaggcagctggctccctctctctgccctgcagggcttgccgcagggcgtgaggctccccagccacagccccctcttacttgccttaaaactggagagaggaatctagtatttgcacttagcaaaatatttaaaaactgaaaaaaatgtgcatgatctgtcactttgtataaaaacaagaaacgattagttcattaaactttttctttaccttctggtagtaaaaatagataaaataattccgtttcaactactgtgtgtaaaaagcctgtatcatatgtaactgggacttttgtaaataaatgtttgtgcgctctgctccctccaaaaaaaaaaaaaaaaaa

SEQ ID NO: 7 - DNA, 25 bp, Artificial Sequence
  Description of Artificial Sequence Synthetic oligonucleotide
cagaaaatagtttgtgcttacacat

SEQ ID NO: 8 - DNA, 25 bp, Artificial Sequence
  Description of Artificial Sequence Synthetic oligonucleotide
tacccgaaggaatttgcgcaccatt

SEQ ID NO: 9 - DNA, 23 bp, Artificial Sequence
  Description of Artificial Sequence Synthetic primer
ggatgactacggtgaatttctgg

SEQ ID NO: 10 - DNA, 26 bp, Artificial Sequence
  Description of Artificial Sequence Synthetic primer
cacttggcttagtgaattcagagatt

SEQ ID NO: 11 - DNA, 15 bp, Artificial Sequence
  Description of Artificial Sequence Synthetic probe
tcttgggcctcctgg

SEQ ID NO: 12 - DNA, 14 bp, Artificial Sequence
  Description of Artificial Sequence Synthetic probe
tcttggcctcctgg

SEQ ID NO: 13 - DNA, 26 bp, Artificial Sequence
  Description of Artificial Sequence Synthetic primer
tggttagatcatccttacttccagtc

SEQ ID NO: 14 - DNA, 23 bp, Artificial Sequence
  Description of Artificial Sequence Synthetic primer
gaggaacgacctggtttgttatt

SEQ ID NO: 15 - DNA, 15 bp, Artificial Sequence
  Description of Artificial Sequence Synthetic probe
tgaaccaacagcccc

SEQ ID NO: 16 - DNA, 15 bp, Artificial Sequence
  Description of Artificial Sequence Synthetic probe
tgaaccaatagcccc

SEQ ID NO: 17 - protein, 50 aa, Homo sapiens
KDDIKYRTSIEEKMTAARIRKCHKCGTGLIKSEGCNRMSCRCGAQMCYLC

SEQ ID NO: 18 - protein, 50 aa, Canis lupus; identical to SEQ ID NO: 17

SEQ ID NO: 19 - protein, 50 aa, Bos taurus
KDDIKYRTSIEEKMTAARIRKCHKCGTSLIKSEGCNRMSCRCGAQMCYLC

SEQ ID NO: 20 - protein, 50 aa, Mus musculus; identical to SEQ ID NO: 17

SEQ ID NO: 21 - protein, 50 aa, Danio rerio
RDEIRLRVAFEEKMTAARVRKCHKCATGLVKSEGCNRMWCRCGAYMCYLC

SEQ ID NO: 22 - protein, 50 aa, Xenopus laevis
SDDVNYRTFIEEKMTAACVRKCHICATELIKAEGCNRLSCRCGAQICYLC

(19) United States
(12) Patent Application Publication
(10) Pub. No.: US 2015/0362491 A1
(43) Pub. Date: Dec. 17, 2015

(54) METHODS AND ASSAYS RELATING TO RNF216

(71) Applicants: Nico KATSANIS, (US); THE GENERAL HOSPITAL CORPORATION, Boston, MA (US)

(72) Inventors: Stephanie B SEMINARA, West Newton, MA (US); Nico KATSANIS, Durham, NC (US)

(73) Assignee: THE GENERAL HOSPITAL CORPORATION, BOSTON, MA (US)

(21) Appl. No.: 14/760,805

(22) PCT Filed: Jan. 14, 2014
(86) PCT No.: PCT/US14/11410

§ 371 Date: Jul. 14, 2015

Related U.S. Application Data
(60) Provisional application No. 61/752,085, filed on Jan. 14, 2013.

(51) Int. Cl. G01N 33/573; A61K 31/56; A61K 31/7048; C12Q 1/68; A61K 31/26

(52) U.S. Cl. CPC G01N 33/573; C12Q 1/6883; A61K 31/26; A61K 31/7048; A61K 31/56; C12Q 2600/156; G01N 2333/9015; G01N 2800/2814

(57) ABSTRACT

The technology described herein relates to the diagnosis and treatment of Gordon Holmes Syndrome and cortical degradation.

### CROSS-REFERENCE TO RELATED APPLICATIONS

This application claims benefit under 35 U.S.C. §119(e) of U.S. Provisional Application No. 61/752,085 filed Jan. 14, 2013, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 14, 2014, is named 030258-076421-PCT_SEtxt and is 54,666 bytes in size.

### TECHNICAL FIELD

The technology described herein relates to methods and assays relating to RNF216 and OTUD4.

### BACKGROUND

A causal genetic locus has not been identified for approximately 40% of cases of cerebellar ataxia, including cases associated with a failure of the reproductive axis, most typically, hypogonadotropic hypogonadism (Sailer and Houlden Curr Neurol Neurosci Rep 2012:227-236; Holmes. Brain 1907:466-489). Strikingly, the genes known to be associated with ataxia have little overlap with genes associated with hypogonadotropic hypogonadism (Balasubramanian and Crowley. Mol Cell Endocrinol 2011 346:4-12). Diagnosis and treatment of cerebellar ataxia in combination with hypogonadotropic hypogonadism could benefit from the elucidation of the underlying pathological genetic defects.

### SUMMARY

As described herein, the inventor has identified muations in RNF216 (a ubiquitin E3 ligase) and OTUD4 (a deubiquitinase) which contribute to Gordon Holmes syndrome, e.g. cerebellar ataxia in combination with failure of the reproductive axis (e.g. hypogonadotropic hypogonadism). The identification of these genes and the specific mutations described herein underlie the assays and methods of treatment described herein, which are directed to the diagnosis and treatment of Gordon Holmes syndrome.

Furthermore, as described herein, the inventor's discovery links disordered ubiquitination to neurodegeneration and reproductive dysfunction. This discovery provides the theoretical underpinnings of the methods of screening described herein, which aim to identify modulators of the RNF216/OTUD4 pathway, e.g. for the treatment of reproductive or sex hormone-dependent conditions.

In one aspect, described herein is a method of diagnosing Gordon Holmes syndrome in a subject, the method comprising: detecting, in a sample obtained from a subject, the presence of a deleterious mutation in RNF216 and optionally, OTUD4; diagnosing the subject with Gordon Holmes syndrome. In some embodiments, the subject is homozygous for the deleterious mutation. In some embodiments, the deleterious mutation results in a truncation of the polypeptide as compared to SEQ ID NO: 1 or SEQ ID NO: 2. In some embodiments, the detecting comprises detecting the size of the RNF216 and optionally, OTUD4 polypeptide. In some embodiments, the deleterious mutation results in a polypeptide which differs by at least 10% from a polypeptide having the sequence of SEQ ID NO: 1 or SEQ ID NO: 2. In some embodiments, the detecting comprises determining the amino acid sequence of the RNF216, and optionally, OTUD4 polypeptide. In some embodiments, the detecting comprises determining the nucleic acid sequence of a nucleic acid molecule encoding RNF216 and optionally, OTUD4. In some embodiments, the deleterious mutation of RNF216 is selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and mutation of R717. In some embodiments, the deleterious mutation of RNF216 is selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C. In some embodiments, the deleterious mutation of OTUD4 is selected from the group consisting of: 998G>T and G333V.

In one aspect, described herein is a method of treating a subject, the method comprising: detecting, in a sample obtained from a subject, the presence of a deleterious mutation in RNF216 and optionally, OTUD4; and administering a treatment for Gordon Holmes syndrome to the subject. In some embodiments, the treatment is increases the expression or activity of RNF216 and optionally, OTUD4. In some embodiments, the treatment is selected from the group consisting of: an ubiquitin acting drug; a drug acting within the proteasome; a drug acting within the ataxia protein protein interaction network; a USP14 inhibitor; IU1; calcium channel blockers; pioglitazone; conenzyme Q10; idebenone; overexpression of heat shock proteins; inhibitors of ATXN1; zolpidem; varenicline; implantation of syngenic cerebellar, gonadotroph, or hypothalaus cells; and implantation of genetically-modified cerebellar, gonadotroph, or hypothalaus cells.

In one aspect, described herein is an assay comprising; detecting, in a sample obtained from a subject, the presence of a deleterious mutation in RNF216 and optionally, OTUD4. In some embodiments, the detecting further comprises measuring whether the subject is homozygous or heterozygous for the deleterious mutation. In some embodiments, the deleterious mutation results in a truncation of the polypeptide as compared to SEQ ID NO: 1 or SEQ ID NO: 2. In some embodiments, the detecting comprises detecting the size of the RNF216 and optionally, OTUD4 polypeptide. In some embodiments, the deleterious mutation results in a polypeptide which differs by at least 10% from a polypeptide having the sequence of SEQ ID NO: 1 or SEQ ID NO: 2. In some embodiments, the detecting comprises determining the amino acid sequence of the RNF216, and optionally, OTUD4 polypeptide. In some embodiments, the detecting comprises determining the nucleic acid sequence of a nucleic acid molecule encoding RNF216 and optionally, OTUD4. In some embodiments, the deleterious mutation of RNF216 is selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and mutation of R717. In some embodiments, the deleterious mutation of RNF216 is selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C. In some embodiments, the deleterious mutation of OTUD4 is selected from the group consisting of: 998G>T and G333V.

In one aspect, described herein is a method of screening a candidate agent which modulates the RNF216/OTUD4 pathway, the method comprising; contacting a cell or organism with a candidate agent; measuring the modulation of a marker of the level of RNF216/OTUD4 pathway activity; wherein candidate agent is identified as an agent which modulates the RNF216/OTUD4 pathway when there is a statistically significant modulation of at least one marker. In some embodiments, the organism is a zebrafish.

In some embodiments, the marker of RNF216/OTUD4 pathway activity is selected from the group consisting of: size of the optic tectum; organization of axonal tracts in the cerebellum; ophthalmic size; the presence or absence of an acerebellar phenotype; Nf-κB pathway activity; GnRH secretion; gonadotrophin secretion; ubiquitination activity or markers; proteasome activity or markers, and the ataxia protein-protein interaction network. In some embodiments, a statistically significant modulation selected from the group consisting of: increase in the size of the optic tectum; increased organization of axonal tract in the cerebellum; increase in ophthalmic size; decrease of acerebellar phenotypes; decreased Nf-κB pathway activity; increased ubiquitination activity or markers; increased proteasome activity or markers; and modulation of the ataxia protein-protein interaction network indicates the candidate agent is an agonist of the RNF216/OTUD4 pathway. In some embodiments, the cell or organism comprises a deleterious RNF216 mutation, and optionally, a deleterious OTUD4 mutation. In some embodiments, the agonist of the RNF216/OTUD4 pathway is identified as a therapeutic agent for a condition selected from the group consisting of: Alzheimer's; Parkinson's disease; ataxia; cerebellar ataxia; neurodegenerative diseases; Gordon Holmes syndrome; and reproductive diseases involving but not limited to hypothalamic and/or pituitary dysfunction. In some embodiments, the reproductive disease involving hypothalamic or pituitary dysfunction is selected from the group consisting of: hypogonadotropic hypogonadism; delayed puberty; amenorrhea; irregular menstrual function; polycystic ovary syndrome; erectile dysfunction; decreased libido; azoospermia; and infertility.

In some embodiments, a statistically significant modulation selected from the group consisting of: decrease in the size of the optic tectum; decreased organization of axonal tract in the cerebellum; decrease in ophthalmic size; increase of acerebellar phenotypes; increased Nf-κB pathway activity; decreased GnRH secretion; and decreased gonadotrophin secretion; decreased ubiquitination activity or markers; decreased proteasome activity or markers, and modulation of the ataxia protein-protein interaction network indicates the candidate agent is an antagonist of the RNF216/OTUD4 pathway. In some embodiments, the antagonist of the RNF216/OTUD4 pathway is identified as a therapeutic agent for a condition selected from the group consisting of: precocious puberty; fibroids; endometriosis; polycystic ovary syndrome; sex-steroid-dependent cancer; breast cancer; and prostate cancer. In some embodiments, the antagonist of the RNF216/OTUD4 pathway is identified as an agent for use in contraception or in vitro fertilization. In some embodiments, the antagonist of the RNF216/OTUD4 pathway is identified as an agent that can induce a state of hypogonadotropic hypogonadism.

### BRIEF DESCRIPTION OF THE DRAWINGS

FIG. 1 depicts graphical representations of the segregation of RNF216 and OTUD4 mutations in the index pedigree and identification of additional RNF216 mutations in unrelated probands. Illustration of the seven-generation pedigree in which Subjects 1-3 presenting with ataxia, dementia and hypogonadotropic hypogonadism are shown to carry both RNF216 p.R751C and OTUD4 p.G333V in homozygosity. Genotyped unaffected family members are shown to be either homozygous for the wt alleles (denoted with a + sign) or heterozygous for one or both changes. In the right half of the figure the families of unrelated RNF216 mutation-positive subjects (Subjects 4-8) with Gordon Holmes syndrome are shown.

FIGS. 2A-2D demonstrate the neuroradiologic and neuropathological findings described herein. FIG. 2A depicts Sagittal T1 or T2-weighted magnetic resonance images of Subjects 3, 4, 6, and 7 reveal diffuse cerebellar and cortical atrophy. FIG. 2B depicts transverse fluid-attenuated inversion recovery (FLAIR) images which reveal multiple distinct and confluent foci of white-matter hyperintensity. FIG. 2C depicts images of immunohistochemical analysis of hippocampal neurons of Subject 2 which reveal intranuclear inclusions that exhibit immunoreactivity with an anti-ubiquitin antibody. FIG. 2D depicts images of electron microscopy of hippocampal neurons of Subject 2 again demonstrates intranuclear inclusions that consist of aggregates of granular material and fine filaments (10-15 nm diameter) that are mostly randomly oriented. Scale bars correspond to 1 μm. The area in the white box is showed at higher magnification in the lower right panel.

FIG. 3 depicts graphs of endocrine phenotypes. Left, Neuroendocrine phenotyping with frequent blood sampling. Arrowheads indicate pulses of luteinizing hormone secretion; boxes are used to show times of sleep. Shaded areas indicate reference ranges for male LH [REFXXX]. Right, Gonadotropin responses to exogenous gonadotropin-releasing hormone across 7 days of receiving pulsatile GnRH. Note the different Y-axis scale for Subject 6. Shaded areas indicate reference ranges [REFSXXX Filicori 86, Hall 92] For Subject 8, the dose-response curve to exogenous GnRH is shown. The solid and dashed lines indicate the mean and 95% confidence interval for six men with hypogonadotropic hypogonadism. Graphs for Subjects 1 and 2 adapted from ref 12. For Subject 6, the dose of GnRH on Day 1 of the first study was 165 ng/kg; all other doses were 75 ng/kg.

FIGS. 4A-4E demonstrate that the RNF216 p.R751C mutation is a loss-of-function allele. FIGS. 4A-4D depict images of dorsal views of the indicated control embryos and embryos injected with rnf216 MO, rnf216 MO+wt human RNF216 and rnf216 MO+mut human RNF216 (RNF216 carrying the p.R751C mutation identified in the index pedigree) at 3 d.p.f. stained with anti-α acetylated tubulin. The dashed ellipse in the control embryo panel (4A) highlights the area of one of the optic tecta, the structure on which all measurements were performed. FIG. 4E depicts a bar graph showing the relative size of the optic tecta in control embryos and embryos injected with rnf216 MO, rnf216 MO+wt human RNF216 and rnf216 MO+mut human RNF216 in arbitrary units (a.u.). Data are presented as mean±s.e. Two-tailed t-tests were performed between the control measurements and each of the rnf216 MO, rnf216 MO+wt human RNF216 and rnf216 MO+mut human RNF216 as well as between the rescue injections with wt and mutant human mRNA. **P-value<0.005 ***P-value<0.0001.

FIGS. 5A-5K demonstrate the epistatic effects of the OTUD4 p.G333V allele. FIGS. 5A-5F depict images of dorsal views of the indicated control embryos and embryos injected with rnf216 MO, otud4 MO, double MO (rnf216 MO+otud4 MO), double MO+wt human OTUD4 and double MO+mut human OTUD4 (OTUD4 carrying the p.G333V mutation identified in the index pedigree) stained with anti-α acetylated tubulin at 3 d.p.f. The stars indicate the optic tecta measured to assess differences between the conditions evaluated. FIG. 5G depicts a graph of the relative sizes of the optic tecta in control embryos and embryos injected with rnf216 MO, otud4 MO, double MO, double MO+wt human RNF216 double MO+mut human RNF216, double MO+wt human OTUD4 and double MO+mut human OTUD4 in arbitrary units (a.u.). Data are presented as mean±s.e. For statistical analyses two-tailed t-tests were performed. *P-value<0.05 **P-value<0.005 ***P-value<0.0001. FIGS. 5H-5J depict images of dorsal views of the indicated control embryos and embryos injected with double MO and double MO+wt human OTUD4 stained with anti-α acetylated tubulin at 3 d.p.f. The dashed boxes indicate the cerebellar areas where maximum disorganization is observed in embryos injected with double MO (I). FIG. 5K depicts a bar graph demonstrating the percentage of embryos with cerebellar defects among the evaluated conditions.

FIG. 6 depicts a schematic illustration of ubiquitination.

FIGS. 7A-7B depict schematic representation of the RNF216 gene and protein. FIG. 7A depicts an illustration of the chromosomal locus harboring the RNF216 gene, where the genomic region is depicted as a horizontal line and the exons as vertical lines. The lines below the genomic representation of the locus depict the normal splicing pattern followed in order to produce the wt human reference protein. A schematic of the protein structure of RNF216 is also shown. The relative position of mutations identified in RNF216 mutation-positive patients are shown with arrows along the protein sequence. The protein domains corresponding to the two RING (really interesting new gene) finger domains and an IBR (in-between RING) domain are given as boxed areas over which the amino-acid positions corresponding to each domain are denoted. FIG. 7B depicts a schematic of the conservation of RNF216 residues R717 and R751 (highlighted) across vertebrate species. (SEQ ID NOS 17-22, respectively, in order of appearance).

FIGS. 8A-8B demonstrate the efficiency of the rnf216 and otud4 morpholinos. FIG. 8A depicts a gel of the results of amplification of the 673 base pair (bp) rnf216 fragment spanning exons 2-7 and surrounding the exon-intron junction targeted by the rnf216 MO reveals retention of intron 2 (77 bp in size) in the MO injected (6 ng) embryos (lanes 2-5). FIG. 8B depicts a gel of the results of injection of the otud4 MO (4-10 ng), which reduces normal splicing of the endogenous transcript by 90% and favors aberrant splicing resulting in a transcript where intron 5 (73 bp in size) is retained.

FIGS. 9A-9E demonstrate that combinatorial knock-down of rnf216 and otud4 induces micropthalmia that can be rescued by co-injection with wt human OTUD4. FIGS. 9A-9D depict images of lateral views of the indicated control embryos and embryos injected with double MO (rnf216 MO, otud4 MO), double MO+wt human OTUD4 and double MO+mut human OTUD4. FIG. 9E depicts a graph of measurements of the eye size area in control embryos and embryos injected with rnf216 MO, otud4 MO, double MO, double MO+wt human RNF216 double MO+mut human RNF216, double MO+wt human OTUD4 double MO+mut human OTUD4 in arbitrary units (a.u.). Data are presented as mean±s.e. Two-tailed t-tests were performed for statistical analyses. *P-value<0.05 ***P-value<0.0001

FIGS. 10A-10E demonstrate treatment of rnf216 and rnf216+otud4 (DMO) with SFN. FIGS. 10A-10C demonstrate treatment with the indicated compounds. Treatment rescues the optic tectum phenotype (FIG. 10D) but not the cerebellar degeneration phenotype (FIG. 10E). *=p-value<0.01; **=p-value<0.001; ***=p-value<0.0001

### DETAILED DESCRIPTION

Embodiments of the technology described herein relate to the inventor's discovery that mutations in RNF216 (an ubiquitin E3 ligase) and OTUD4 (a deubiquitinase) can cause Gordon Holmes syndrome, e.g. cerebellar ataxia in combination with failure of the reproductive axis (e.g. hypogonadotropic hypogonadism). Accordingly, described herein are methods of diagnosing and treating Gordon Holmes syndrome.

Furthermore, as described herein, the inventor's discovery additionally relates to the finding that modulation of ubiquitination, caused by perturbation of the RNF1216/OTUD4 pathway, leads to modulation of neuronal organization and health as well as reproductive function. Accordingly, described herein are methods of screening, relating to the identification of modulators of the RNF216/OTUD4 pathway, e.g. for the treatment of neurodegenerative and/or reproductive or sex hormone-dependent conditions.

In one aspect, described herein is a method of diagnosing Gordon Holmes syndrome in a subject, the method comprising: detecting, in a sample obtained from a subject, the presence of a deleterious mutation in RNF216 and optionally, OTUD4; and diagnosing the subject with Gordon Holmes syndrome.

As used herein, “Gordon Holmes syndrome” refers to a condition characterized by ataxia and hypogonadism. The hypogonadism is typically hypogonadotropic, but may be hypergonadotropic. Dementia often occurs. Subjects can exhibit delayed puberty, hypogonadism, infertility, impaired cognitive function, dementia, lack of coordination, tremors, amenorrhea, and/or cessation of menses. Symptoms may develop at any point, with adult-onset being known. Tests and assays that can be helpful in making a diagnosis of Gordon Holmes syndrome can include, brain imaging, assays for sex hormone and gonadotropin levels, and measures of ataxia.

As used herein, “ring finger protein 216” or “RNF216” (also known as TRIAD3 and ZIN) refers to an E3 ubiquitin ligase which operates within, but not exclusively, the NF-κB pathway. Overexpression of RNF216 can inhibit NF-κB, presumably via ubiquitination and proteasomal degradation of other proteins. Accordingly, deleterious mutations in RNF216 can perturb the NF-κB pathway. The sequence of RNF216 expression products for a number of species is well known in the art, e.g. human RNF216 (e.g. human RNF216 protein (SEQ ID NO: 1, NCBI Ref Seq: NP_{—}996999 and SEQ ID NO: 5, NCBI Ref Seq: NP_{—}996994) and the human RNF216 mRNA (SEQ ID NO: 3, NCBI Ref Seq: NM_{—}207116 and SEQ ID NO: 6; NCBI Ref Seq: NM_{—}207111) (NCBI Gene ID: 54476).

As used herein, “ovarian tumor (OTU) domain containing 4” or “OTUD4” refers to deubiquitinating enzyme (DUB). In some embodiments, OTUD4 can, interact with and/or regulate the ubiquitination activity of RNF216. In some embodiments, deleterious mutations in OTUD4 can perturb, e.g. the NF-κB pathway and sex hormone production, in some cases in epistatic interactions with deleterious mutations of RNF216. The sequence of OTUD4 expression products for a number of species is well known in the art, e.g. human OTUD4 (e.g. human OTUD4 protein (SEQ ID NO: 2, NCBI Ref Seq: NP_{—}001096123) and the human OTUD4 mRNA (SEQ ID NO: 4, NCBI Ref Seq: NM_{—}001102653) (NCBI Gene ID: 54726).

As described herein, in some embodiments, mutations of RNF216 alone are sufficient to cause disease. In some embodiments, mutations of RNF216 exhibit epistatic interactions with mutations of OTUD4 to cause disease. Accordingly, in some embodiments, the presence of only a deleterious mutation of RNF216 can be detected. Alternatively, in some embodiments, the presence of deleterious mutations of RNF216 and OTUD4 can be detected.

As used herein, a “deleterious mutation” is a mutation which reduces the level and/or activity of a given gene. For example, a deleterious mutation can include, but is not limited to, a mutation in the promoter of a gene which reduces expression, a nonsynonymous mutation, a missense mutation, a mutation which results in premature termination, a mutation which decreases enzymatic activity (e.g. a mutation which alters the enzymatic active site), and/or a mutation which alters substrate specificity (e.g. a mutation which alters substrate recognition and/or binding specificity). In some embodiments, a deleterious mutation of RNF216 and/or OTUD4 can be a mutation which results in a reduction of the level and/or activity of the polypeptide expression product of at least 10%, e.g. a 10% reduction, a 20% reduction, a 30% reduction, a 40% reduction, a 50% reduction, a 60% reduction, a 70% reduction, a 80% reduction, a 90% reduction, or more.

In some embodiments, a deleterious mutation of RNF216 and/or OTUD4 can be a mutation which results in a truncation of the polypeptide expression product as compared to, respectively, SEQ ID NO: 1 or SEQ ID NO: 2. In some embodiments, a deleterious mutation of RNF216 and/or OTUD4 can be a mutation which results in a truncation of the polypeptide expression product as compared to, respectively, SEQ ID NO: 1 (or SEQ ID NO: 5) or SEQ ID NO: 2 by at least 10%, e.g. 10% or more truncation, 20% or more truncation, 30% or more truncation, 40% or more truncation, 50% or more truncation, 60% or more truncation, 70% or more truncation, 80% or more truncation, or 90% or more truncation.

In some embodiments, a deleterious mutation of RNF216 and/or OTUD4 can be a mutation which causes the polypeptide expression product to differ by at least 10% from a polypeptide having the sequence of, respectively, SEQ ID NO: 1 (or SEQ ID NO: 5) or SEQ ID NO: 2, e.g. a mutation which cause the expression product to differ by 10%, 20%, 30%, 40%, 50%, 60%, 70%, 80%, 90% or more.

In some embodiments, the deleterious mutation of RNF216 can be selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and/or mutation of R717.

In some embodiments, the deleterious mutation of RNF216 is selected from the group consisting of: c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C.

In some embodiments, the deleterious mutation of OTUD4 can be selected from the group consisting of: c.998G>T and p.G333V.

In some embodiments, a subject can be diagnosed if they are homozygous for the deleterious mutation. In some embodiments, a subject with Gordon Holmes syndrome is homozygous for the deleterious mutation.

In one aspect, the technology described herein relates to an assay comprising; detecting, in a sample obtained from a subject, the presence of a deleterious mutation in RNF216 and optionally, OTUD4.

In some embodiments, the detecting comprises determining the nucleic acid sequence of a nucleic acid molecule encoding RNF216 and optionally, a nucleic acid molecule encoding OTUD4. For example, the presence of the mutation can be determined using an assay selected from the group consisting of: hybridization; sequencing; exome capture; PCR; and high-throughput sequencing. In some embodiments the mutation is present in genomic DNA. In some embodiments, the mutation is present in mRNA transcripts.

In some embodiments, detecting comprises determining the amino acid sequence of the RNF216, and optionally, OTUD4 polypeptide, e.g. by sequencing the polypeptides (e.g. by mass spectrometry) or by the use of reagents specific for particular polypeptide sequences, e.g. antibody reagents specific for polypeptides comprising a deleterious mutation as described herein.

In some embodiments, detecting comprises detecting the size of the RNF216 and optionally, OTUD4 polypeptide, e.g. by PAGE analysis of polypeptides recognized by, e.g. an RNF216-specific antibody reagent.

In some aspects, described herein is an assay comprising: contacting a sample obtained from a human subject having cancer with a probe to detect the presence of a deleterious mutation in RNF216 and optionally, OTUD4; and detecting the presence or intensity of a signal which indicates the presence of a deleterious mutation in RNF216 and optionally, OTUD4. In some embodiments of the foregoing aspects, a detectable signal is generated by the probe when a deleterious mutation is present. In some embodiments of the foregoing aspects, the probe is detectably labeled. In some embodiments, the probe can be a nucleic acid probe. In some embodiments, the probe can be an antibody or antibody reagent probe.

In some embodiments of the foregoing aspects, the method can further comprise the step of generating a report based on the detection of a deleterious mutation in RNF216 and/or OTUD4 by a non-human machine.

In some embodiments, the presence of a deleterious mutation, e.g. in RNF216 and/or OTUD4 can be detected by determining the sequence of a genomic locus and/or an mRNA transcript. Such molecules can be isolated, derived, or amplified from a biological sample. Nucleic acid (e.g. DNA) and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; and proteinase K extraction can be used to obtain nucleic acid from blood (Roiff, A et al. PCR: Clinical Diagnostics and Research, Springer (1994)).

In some embodiments, the nucleic acid sequence of a target gene (e.g. RNF216 and/or OTUD4) in a sample obtained from a subject can be determined and compared to a reference sequence to determine if a deleterious mutation is present in the subject. In some embodiments, the sequence of the target gene can be determined by sequencing the target gene (e.g. the genomic sequence and/or the mRNA transcript thereof). Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (3 ed.), Cold Spring Harbor Laboratory Press, Cold Spring Harbor, N.Y., USA (2001); which are incorporated by reference herein in their entireties.

In some embodiments, sequencing can comprise exome sequencing (i.e. targeted exome capture). Exome sequencing comprises enriching for an exome(s) of interest and then sequencing the nucleic acids comprised by the enriched sample. Sequencing can be according to any method known in the art, e.g. those described above herein. Methods of enrichment can include, e.g. PCR, molecular inversion probes, hybrid capture, and in solution capture. Exome capture methodologies are well known in the art, see, e.g. Sulonen et al. Genome Biology 2011 12:R94; and Teer and Mullikin. Hum Mol Genet 2010 19:R2; which are incorporated by reference herein in their entireties. Kits for performing exome capture are available commercially, e.g. the TRUSEQ™ Exome Enrichment Kit (Cat. No. FC-121-1008; Illumnia, San Diego, Calif.). Exome capture methods can also readily be adapted by one of skill in the art to enrich specific exomes of interest.

In some embodiments, the presence of a deleterious mutation can be determined using a probe that is specific for the deleterious mutation. In some embodiments, the probe can be detectably labeled. In some embodiments, a detectable signal can be generated by the probe when a deleterious mutation is present. In some embodiments, the probe specific for the deleterious mutation can be a probe in a hybridization assay, i.e. the probe can specifically hybridize to a nucleic acid comprising a deleterious mutation (as opposed to a wild-type nucleic acid sequence) and the hybridization can be detected, e.g. by having the probe and or the target nucleic acid be detectably labeled. Hybridization assays are well known in the art and include, e.g. northern blots and Southern blots.

In some embodiments, the probe specific for the deleterious mutation can be a probe in a PCR assay, i.e. a primer. In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a thermostable DNA polymerase, and optionally, (iii) screening the PCR products for a band or product of the correct size. The primers used are oligonucleotides of sufficient length and appropriate sequence to provide initiation of polymerization, i.e. each primer is specifically designed to be complementary to a strand of the genomic locus to be amplified. In an alternative embodiment, the presence of a deleterious mutation in an mRNA transcript can be determined by reverse-transcription (RT) PCR and by quantitative RT-PCR (QRT-PCR) or real-time PCR methods. Methods of RT-PCR and QRT-PCR are well known in the art. In some embodiments, the PCR product can be labeled, e.g. the primers can comprise a detectable label, or a label can be incorporated and/or bound to the PCR product, e.g. EtBr detection methods.

In addition, protein sequences and/or sizes may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000). In certain embodiments, a gas phase ion spectrophotometer is used.

In other embodiments, laser-desorption/ionization mass spectrometry is used to analyze the level of a protein. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. The mass spectrometers and their techniques are well known to those of skill in the art.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience, e.g. a change in color, upon encountering the targeted molecules or upon treatment with a chemical agent. In some instances, signal amplification can be integrated into the particular protocol, wherein a secondary antibody, that includes the marker signal or marker activity (e.g. an enzyme activity), follows the application of a primary target-specific antibody.

In some embodiments, the methods and assays described herein can comprise a detection agent specific for an expression product of RNF216 and/or OTUD4 comprising at least one deleterious mutation. A detection agent can be any agent which can specifically detect the presence of the target (e.g. bind specifically to the target) according to an assay described herein, e.g. a detection reagent can be a nucleic acid probe or primer specific for the target or an agent which specifically binds to a target polypeptide (e.g. a polypeptide comprising a deleterious mutation of RNF216 and/or OTUD4). In some embodiments, the detection reagent can comprise a detectable signal or be capable of generating a detectable signal. In some embodiments, the detection agent can be an antibody reagent. In some embodiments, the detection agent can be a monoclonal antibody and/or comprise CDRs of a monoclonal antibody.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeable herein are used herein to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. For example, a reagent can bind to a polypeptide and/or nucleic acid comprising a deleterious mutation of RNF216 (e.g. a deleterious mutant polypeptide of RNF216) and/or OTUD4 with greater specificity and affinity than a polypeptide and/or nucleic acid not comprising a deleterious mutation of RNF216 and/or OTUD4 (e.g. wildtype RNF216 polypeptide).

The term “label” refers to a composition capable of producing a detectable signal indicative of the presence of an antibody reagent (e.g. a bound antibody reagent). Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

Provided herein are methods comprising detecting the presence of a deleterious mutation in a subject. Such a measurement can be made, for example, on a representative tissue sample or other form of biological sample, obtained from the subject. In one embodiment, the cell or tissue is obtained from the subject in the form of a biological sample. The appropriate detection is then performed on the biological sample. When necessary, the biological sample can be further processed. For example, a bodily fluid obtained from the subject can be further processed to purify or further concentrate cells or tissues therein.

The term “biological sample” as used herein denotes a sample taken or isolated from a biological organism, e.g., biopsy sample, tissue cell culture supernatant, cell lysate, lymph, a homogenate of a tissue sample from a subject or a fluid sample from a subject. Exemplary biological samples include, but are not limited to, tissue biopsies, hypothalamus tissue, cerebellar tissue, gonadotroph tissue, and/or bodily fluids such as blood, lymph, etc. In some embodiments, the sample is normal tissue.

The term “biological sample” also includes untreated or pretreated (or pre-processed) biological samples. In some embodiments, the biological sample is an untreated biological sample. As used herein, the phrase “untreated biological sample” refers to a biological sample that has not had any prior sample pre-treatment except for dilution and/or suspension in a solution. Exemplary methods for treating a biological sample include, but are not limited to, centrifugation, filtration, sonication, homogenization, heating, freezing and thawing, and any combinations thereof. The skilled practitioner is aware of methods and processes appropriate for pre-processing of biological samples required for determination of levels of proteins as described herein.

The sample can be obtained by removing a sample of cells from a subject, but can also be accomplished by using previously isolated cells (e.g. isolated at a prior time point and isolated by the same or another person). In addition, the biological sample can be freshly collected or a previously collected sample. In some embodiments, a biological sample is a biological fluid. Examples of biological fluids include, but are not limited to, saliva, blood, sputum, an aspirate, and any combinations thereof.

In some embodiments, the biological sample is a frozen biological sample, e.g., a frozen tissue or fluid sample such as sputum. The frozen sample can be thawed before employing methods, assays and systems of the invention. After thawing, a frozen sample can be centrifuged before being subjected to methods, assays and systems of the invention. In some embodiments, the biological sample can be treated with at least one chemical reagent, such as a protease inhibitor. In some embodiments, the biological sample is a clarified biological sample, for example, by centrifugation and collection of a supernatant comprising the clarified biological sample. In some embodiments, a biological sample is a pre-processed biological sample, for example, supernatant or filtrate resulting from a treatment selected from the group consisting of centrifugation, filtration, sonication, homogenization, lysis, thawing, amplification, purification, restriction enzyme digestion ligation and any combinations thereof.

In some embodiments, the biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

In one aspect, the technology described herein relates to a method of treating a subject, the method comprising: detecting, in a sample obtained from a subject, the presence of a deleterious mutation in RNF216 and optionally, OTUD4; and administering a treatment for Gordon Holmes syndrome to the subject.

In some embodiments, the treatment for Gordon Holmes syndrome can increase the expression or activity of RNF216 and optionally, OTUD4. By way of non-limiting example, the subject can be administered polypeptides comprising SEQ ID NO:1 and/or SEQ ID NO:2, and/or variants thereof or nucleic acids encoding such polypeptides. In some embodiments, the level of RNF216 and/or OTUD4 in a subject is increased by administering a composition comprising a RNF216 and/or OTUD4 polypeptide and/or a nucleic acid encoding a RNF216 and/or OTUD4 polypeptide. A RNF216 and/or OTUD4 polypeptide administered to a subject according to the methods described herein can comprise a RNF216 and/or OTUD4 polypeptide as described herein above.

In some embodiments, a RNF216 and/or OTUD4 polypeptide as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a RNF216 and/or OTUD4 polypeptide as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, a RNF216 and/or OTUD4 polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a RNF216 and/or OTUD4 polypeptide can be modified, e.g. by addition of a moiety to one or more of the amino acids comprising the peptide. In some embodiments, a RNF216 and/or OTUD4 polypeptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, a RNF216 and/or OTUD4 polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a RNF216 and/or OTUD4 polypeptide can be increased by the addition of moieties, e.g. PEG or albumin.

In some embodiments, the RNF216 and/or OTUD4 polypeptide administered to the subject can be a functional fragment of one of the RNF216 and/or OTUD4 amino acid sequences described herein. As used herein, a “functional fragment” is a fragment or segment of a peptide which can serve as an agonist of the RNF216/OTUD4 pathway according to the assays described below herein. A functional fragment can comprise conservative substitutions of the sequences disclosed herein.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a RNF216 and/or OTUD4 polypeptide as described herein can be chemically synthesized and mutations can be incorporated as part of the chemical synthesis process.

In some embodiments, a RNF216 and/or OTUD4 polypeptide as described herein can be formulated as a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See, e.g. Wang et al. “Prodrug approaches to the improved delivery of peptide drug” in Curr. Pharm. Design. 5(4):265-287 (1999); “Improved passive oral drug delivery via prodrugs”, Adv. Drug Delivery Rev., 19(2): 131-148 (1996); and Waller et al., “Prodrugs”, Br. J. Clin. Pharmac. 28: 497-507 (1989), which are incorporated by reference herein in their entireties.

In some embodiments, a RNF216 and/or OTUD4 polypeptide as described herein can be a pharmaceutically acceptable solvate. The term “solvate” refers to a peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The peptides of the present invention can be synthesized by using well known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, N.Y. (1984); Kimmerlin, T. and Seebach, D. J. Pept. Res. 65:229-260 (2005); Nilsson et al., Annu. Rev. Biophys. Biomol. Struct. (2005) 34:91-118; W. C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2^{nd} Ed, Oxford University Press, Cary, N.C. (2002); and P. Lloyd-Williams, F. Albericio, and E. Giralt, Chemical Approaches to the synthesis of peptides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, the technology described herein relates to a nucleic acid encoding a RNF216 and/or OTUD4 polypeptide as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a RNF216 polypeptide can comprise the nucleotide sequence of SEQ ID NO: 3 or SEQ ID NO: 6. In some embodiments, a nucleic acid encoding a OTUD4 polypeptide can comprise the nucleotide sequence of SEQ ID NO: 4. In some embodiments, a nucleic acid encoding a RNF216 or OTUD4 polypeptide as described herein is comprised by a vector. In some of the aspects described herein, a nucleic acid sequence encoding a polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a RNF216 or OTUD4 polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments the level of RNF216 and/or OTUD4 in the subject is increased by at least 20% over the level of RNF216 and/or OTUD4 in the subject prior to treatment, e.g. 20% or more, 30% or more, 40% or more, 50% or more, 100% or more, 150% or more, 200% or more, 250% or more, 300% or more, or 350% or more. In some embodiments the level of RNF216 and/or OTUD4 in the subject is increased by at least 100% over the level of RNF216 and/or OTUD4 in the subject prior to treatment. In some embodiments the level of RNF216 and/or OTUD4 in the subject is increased by at least 200% over the level of RNF216 and/or OTUD4 in the subject prior to treatment. In some embodiments the level of RNF216 and/or OTUD4 in the subject is increased by about 250% over the level of RNF216 and/or OTUD4 in the subject prior to treatment. In some embodiments, the level of RNF216 and/or OTUD4 in the subject is increased to at least 50% of a healthy reference level, e.g. 50% or more, 60% or more, 70% or more, 80% or more, 90% or more, or 100% or more of a healthy reference level. In some embodiments, the level of RNF216 and/or OTUD4 in the subject is increased to at least 60% of a healthy reference level. In some embodiments, the level of RNF216 and/or OTUD4 in the subject is increased to at least 75% of a healthy reference level. In some embodiments, the level of RNF216 and/or OTUD4 in the subject is increased to at least 90% of a healthy reference level. A healthy reference level can be the average level of RNF216 and/or OTUD4 in a population of human subjects lacking deleterious mutations of RNF216 and/or OUTD4.

In some embodiments, the treatment for Gordon Holmes syndrome can be selected from the group consisting of: molecules that prevent the phosphorylation and/or promote the ubiquitination of IκB; molecules that modulate the NF-κB pathway; an ubiquitin acting drug; molecules that modulate proteasome function; a USP14 inhibitor; IU1; molecules that affect the ataxia protein protein interaction network; calcium channel blockers; pioglitazone; coenzyme Q10; idebenone; overexpression of heat shock proteins; inhibitors of ATXN1; zolpidem; varenicline; implantation of syngenic cerebellar, gonadotroph, or hypothalamus cells; and implantation of genetically-modified cerebellar, gonadotroph, or hypothalamus cells.

In some embodiments, the drug that modulates proteasome function can be a proteasome activator. Examples of proteasome activators are known in the art and can include, by way of non-limiting example, SFN (1-isothio-cyanato-4(R)-methylsulfinylbutane) (e.g. a compound having the structure of Formula I); (methylsulfonyl)cyclohexylmethylisothiocyanante: oleuropein; betulinic acid; and derivatives thereof. The art is familiar with sulforaphane analogs, which include, but are not limited to, the following: 6-isothiocyanato-2-hexanone, exo-2-acetyl-6-isothiocyanatonorbornane, exo-2-isothiocyanate-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanante and trans-3-(methylsulfonyl)cyclohexylmethylisothiocyanante. Methods of synthesizing and/or isolating these compounds are known in the art and/or the compounds are available commercially, e.g. SFN is available from Sigma-Aldrich (Cat No 6317; St. Louis, Mo.). For further discussion of proteasome activators, see, e.g., Huang and Chen. Curr Med Chem 2009 16:931-9; Wilk and Chen. Mol Biol Rep 1997 24:119-124; Ban et al. ChemMedChem 2010 5:1236-1241; each of which is incorporated by reference herein in its entirety.

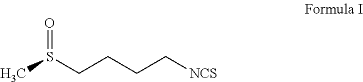

RIP is a signal transducer (serine threonine kinase) that is required for TNF-mediated NF-κB activation. RNF216's other name is ZIN because it is thought to be an inhibitor of RIP-mediated NF-κB activation (zinc finger protein inhibiting NF-κB activation pathways) (Chen D, Li X Zhai Z, Shu H, JBC 2002). As described herein, mutations of RNF216 would be expected to prevent the inhibition of NF-κB activation. Loss of function mutations in RNF216, as described herein, can activate this pathway inappropriately (i.e. in the absence of stress). In some embodiments, Gordon Holmes syndrome can be treated by inhibiting the inappropriate activation of the NF-κB pathway, e.g. by preventing or slowing NF-κB's translocation into the nucleus. By way of non-limiting example, therapeutic agents can include molecules that prevent the phosphorylation IκB (unphosphorylated IκB keeps NF-κB in its inactive form) or molecules that prevent the ubiquitination of IκB (which would prevent the degradation of IκB).

Ubiquitination can serve as a recognition signal for proteasomal degradation (polyubiquitylation), serve as a signaling scaffold for protein-protein interactions (Lys63-poly or monoubiquitylation) or represent a targeting signal for the lysosomal pathway or other cellular components (monoubiquitylation). The ability of the ubiquitylation machinery to selectively target substrates is mediated by the specificity of ubiquitin ligation (E2 and E3 enzymes) and deconjugation, promoted by deubiquitylating enzymes (DUBs). Since both proteins involved in this disease (i.e. RNF216 and OTUD4) modulate ubiquitination, therapeutic agents for the treatment of Gordon Holmes syndrome include molecules that affect ubiquitination. This includes molecules that affect the ubiquitination and de-ubiquitination of RNF216 and OTUD4 themselves. This can also include molecules that modulate the intrinsic activity of the ubiquitination and DUB activity of RNF216 and OTUD4, or that act by restricting or extending the length of ubiquitin chains attached to substrates.

Substrate deubiquitination on the proteasome is mediated by three distinct deubiquitinating enzymes associated with the regulatory particle of the proteasome: RPN11, UCH37, and USP14. Accordingly, therapeutic agents for Gordon Holmes syndrome can include molecules that modulate ubiquitination, including but not limited to, inhibitors against proteasome deubiquitylating enzymes. By way of non-limiting example, the small molecule compound IU1 was found to be a selective inhibitor of USP14. IU1-mediated inhibition of USP14 indirectly accelerates proteasomal degradation of proteins, such as tau and ataxin-3, both of which are involved in neurodegenerative diseases (Lee B et al 2010 Nature). Because neurodegenerative disorders such as Parkinson disease (and possibly also Gordon Holmes syndrome) are associated with the accumulation of misfolded proteins, IU1 or other USP14-directed small molecule inhibitors could potentially be used to eliminate these toxic proteins and improve the prognosis in neurodegenerative diseases.

The ubiquitin-proteasome system deals with misfolded proteins but also has a role in transcription by regulating the assembly and disassembly of transcription complexes. Therefore, it is contemplated that mutations in RNF216 and OTUD4 can regulate gene expression through their ubiquitination activity of certain transcription factors that are critical for establishing correct assembly/disassembly cycle of transcription complexes. Therefore, potential therapies include molecules that regulate the stability of transcription factor complexes targeting by RNF216 and/or OTUD4.

Some Gordon Holmes syndrome patients described herein have mitochondrial defects. It is contemplated that mitochondrial defects occur downstream of loss of RNF216 and OTUD4. These can include dysregulated intracellular and mitochondrial calcium homeostasis, mitochondrial enzyme activity deficits, and modifications of mitochondrial membrane potential. Therefore, therapeutic agents can include those that improve mitochondrial integrity and mitochondrial performance, e.g. calcium channel blockers, iron chelators, deferiprone (see, e.g. Boddaert et al. Blood. 2007; 110(1):401), pioglitazone, Coenzyme Q10, Idebenone (a free radical scavenger; see e.g. Mariotti et al. Neurology. 2003; 60(10):1676; Hausse et al. Heart. 2002; 87(4):346)), and vitamin E (see, e.g. Schulz et al. Neurology. 2000; 55(11):1719; Lodi et al. Ann Neurol. 2001; 49(5):590; Hart et al. Arch Neurol. 2005; 62(4):621). The foregoing references are incorporated herein in their entireties by reference.

The presence of intranuclear inclusions found in the brain of one of the patients as described herein indicates that Gordon Holmes syndrome can involve a disruption of the protein homeostatic network of cellular pathways. Therefore, therapeutic agents can include agents which regulate protein homeostasis. Animal studies suggest that polyglutamine neurotoxicity can be suppressed by overexpression of heat shock proteins (see, e.g. Cummings et al. Nat Genet. 1998; 19(2):148; Kazemi-Esfarjani P, Benzer S Science. 2000; 287(5459):1837; Warrick et al. NM Nat Genet. 1999; 23(4):425). The foregoing references are incorporated herein in their entireties by reference.

Because there appears to be some selective vulnerability of the cerebellum and pituitary/hypothalamus, one therapeutic approach is to reduce the expression of the mutant gene(s) in those structures. On approach that has been shown to be effective in reducing the expression of a mutant gene for ataxia (ATXN1) in vivo is delivery to the cerebellum of AAV vectors expressing a shRNA directed against the mutant gene (Xia et al, 2004). Through this approach, mutant ATXN1 expression was reduced and motor function significantly improved and Purkinje cell morphology was normalized in the cells that received the shRNA. This study used a shRNA that targeted both wild type and mutant ATXN1, but there are methods being developed where the mutant allele would be preferentially targeted (Alves et al, 2008). Accordingly, therapeutic agents for Gordon Holmes syndrome can include inhibitory nucleic acids, e.g. shRNAs, RNAi, or siRNA that inhibit expression of deleterious mutants of RNF216 and/or OTUD4.

In certain embodiments, contacting a cell with the inhibitory nucleic acid results in a decrease in the target mRNA level in a cell of at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased by at least about 70%, about 80%, about 90%, about 95%, about 99%, about 100%. In certain embodiments, the inhibitor can comprise an expression vector or viral vector comprising the RNAi molecule.

As used herein, the term “RNAi” refers to any type of interfering RNA, including but are not limited to RNAi, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” and “RNA interfering” with respect to an agent of the technology described herein, are used interchangeably herein.

As used herein a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

RNAi may be delivered with the help of nanoparticles as described for example in Schiffelers and Storm, Expert Opin Drug Deliv. 2006 May; 3(3):445-54 or liposomes (e.g. Hughes et al., Methods Mol Biol. 2010; 605:445-59).

Because of the selective vulnerability of the cerebellum and pituitary gonadotrophs/hypothalamus in this disease, it is contemplated that patient-derived pluripotent stem cell (iPSC) based cell replacement strategies can be therapeutic. Thus, subject with Gordon Holmes syndrome can be treated by the administration of syngenic and/or genetically-modified cerebellar, gonadotroph, or hypothalamus cells. In some embodiments, the cells can be differentiated cells obtained from the subject and genetically modified to express wildtype RNF216 and/or OTUD4 before being administered to the subject. In some embodiments, the cells can be cells obtained from the subject that were dedifferentiated to iPSC phenotypes and genetically modified to express wildtype RNF216 and/or OTUD4 before being administered to the subject, either as pluripotent cells or the at least partially differentiated daughter cells of such pluripotent cells.

In some embodiments, a therapeutic agent for the treatment of Gordon Holmes syndrome can be zolpidem (see, e.g. Clauss R, Sathekge M, Nel W N Engl J Med. 2004; 351(5):511). In some embodiments, a therapeutic agent for the treatment of Gordon Holmes syndrome can be varenicline (see, e.g. Zesiewicz T A, Greenstein P E, Sullivan K L, Wecker L, Miller A, Jahan I, Chen R, Perlman S L Neurology. 2012 February; 78(8):545-50. Epub 2012 February 8.). The foregoing references are incorporated herein in their entireties by reference.

In one aspect, the technology described herein relates to a method of screening a candidate agent which modulates the RNF216/OTUD4 pathway, the method comprising; contacting a cell or organism with a candidate agent; measuring the modulation of a marker of the level of RNF216/OTUD4 pathway activity; wherein candidate agent is identified as an agent which modulates the RNF216/OTUD4 pathway when there is a statistically significant modulation of at least one marker. In some embodiments, the organism can be a zebrafish. As used herein, “RNF216/OTUD4 pathway” refers to the signaling pathway(s) which require the action of RNF216/OTUD4 and the molecules which are a part thereof. That is, signals and/or activities which are perturbed by a modulation of RNF216 and/or OTUD4 are part of the RNF216/OTUD4 pathways. Such signals, levels, and/or activities can be used as markers of the activity of the RNF216/OTUD4 pathway. Non-limiting exemplary markers of the RNF216/OTUD4 pathway include size of the optic tectum; organization of axonal tracts in the cerebellum; ophthalmic size; the presence or absence of an acerebellar phenotype; Nf-κB pathway activity; GnRH secretion; gonadotrophin secretion; ubiquitination activity or markers; ataxia protein-protein interaction activity or markers; and proteasome activity or markers.

As used herein, a “candidate agent” refers to any entity which is normally not present or not present at the levels being administered to a cell, tissue or subject. A candidate agent can be selected from a group comprising: chemicals; small organic or inorganic molecules; nucleic acid sequences; nucleic acid analogues; proteins; peptides; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the candidate agent is any chemical, entity or moiety, including without limitation synthetic and naturally-occurring non-proteinaceous entities. In certain embodiments the candidate agent is a small molecule having a chemical moiety. For example, chemical moieties include unsubstituted or substituted alkyl, aromatic, or heterocyclyl moieties including macrolides, leptomycins and related natural products or analogues thereof. Candidate agents can be known to have a desired activity and/or property, or can be selected from a library of diverse compounds.

Candidate compounds and agents can be screened for their ability to modulate RNF216/OTUD4 pathway markers and/or activity in vitro. The modulation of ClpP1P2 activity can also be monitored in vivo, e.g. in zebrafish as described elsewhere herein. In one embodiment, candidate agents are screened using the assays for RNF216/OTUD4 pathway markers and/or activity described below herein. Candidate agents are typically first screened for their ability to modulate RNF216/OTUD4 pathway activity in vitro and those candidate agents with such modulatory effects are identified. Positive modulatory agents are then tested for efficacy with respect to modulation of RNF216/OTUD4 pathway activity in an in vivo assay.

Generally, compounds can be tested at any concentration that can modulate cellular function, gene expression or protein activity relative to a control over an appropriate time period. In some embodiments, compounds are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM.

Depending upon the particular embodiment being practiced, the candidate or test agents can be provided free in solution, or can be attached to a carrier, or a solid support, e.g., beads. A number of suitable solid supports can be employed for immobilization of the candidate agents. Examples of suitable solid supports include agarose, cellulose, dextran (commercially available as, e.g., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, Mass.), Invitrogen (Carlsbad, Calif.), Ryan Scientific (Mt. Pleasant, S.C.), and Enzo Life Sciences (Farmingdale, N.Y.). These libraries can be screened for the ability of members to modulate RNF216/OTUD4 pathway activity using e.g. methods described herein.

In some embodiments, the candidate agents can be naturally occurring proteins or their fragments. Such candidate agents can be obtained from a natural source, e.g., a cell or tissue lysate. Libraries of polypeptide agents can also be prepared, e.g., from a cDNA library commercially available or generated with routine methods. The candidate agents can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. In some methods, the candidate agents are polypeptides or proteins. Peptide libraries, e.g. combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The candidate agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

In some embodiments, the candidate agent that is screened and identified to modulate RNF216/OTUD4 pathway activity according to the methods described herein by at least 5%, preferably at least 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control.

The candidate agent can function directly in the form in which it is administered. Alternatively, the candidate agent can be modified or utilized intracellularly to produce a form that modulates the desired activity, e.g. introduction of a nucleic acid sequence into a cell and its transcription resulting in the production of an inhibitor or activator of gene expression or protein activity within the cell.

In some embodiments, a candidate agent can cause a statistically significant modulation selected from the group consisting of: increase in the size of the optic tectum; increased organization of axonal tract in the cerebellum; increase in ophthalmic size; decrease of acerebellar phenotypes; decreased Nf-κB pathway activity; increased ubiquitination activity or markers; modulation of the ataxia protein-protein interaction network; and increased proteasome activity or markers; indicating that the candidate agent is an agonist of the RNF216/OTUD4 pathway.

In some embodiments, modulation of GnRH secretion and/or gonadotroph secretion indicates a candidate agent is an agonist of the RNF216/OTUD4 pathway. In a healthy organism, GnRH and gonadotrophin secretion is pulsatile in nature. Agents which cause a sustained secretion of GnRH and gonadotrophin can result in the downstream components of the relevant pathways becoming desensitized to GnRH and/or gonadotrophin secretion, thus ultimately acting as antagonists of sex hormone signaling. Accordingly, in some embodiments, an agonist of the RNF216/OTUD4 pathway can be an agent which induces pulsatile GnRH and/or gonadotrophin secretion. Accordingly, in some embodiments, an agonist of the RNF216/OTUD4 pathway can be an agent which increases the magnitude of pulsatile GnRH and/or gonadotrophin secretion. Accordingly, in some embodiments, an agonist of the RNF216/OTUD4 pathway can be an agent which modulates the frequency of pulsatile GnRH and/or gonadotrophin secretion. Accordingly, in some embodiments, an agonist of the RNF216/OTUD4 pathway can be an agent which modulates pulsatile GnRH and/or gonadotrophin secretion in such a way that downstream signaling partners do not become desensitized.

Methods of measuring the modulation of markers of RNF216/OTUD4 pathway are known in the art. For example, GnRH secretion can be measured, e.g. by measuring the level of GnRH-induced luteinizing hormone (LH) pulse patterns in blood samples, e.g. in zebrafish using, e.g. microparticle enzyme immunoassay (e.g. the AxSYM™ system; Abbott Laboratories, Abbott Park, Ill.). As a further example, cerebellar morphology and organization can be measured by staining cerebellar samples with anti-α acetylated tubulin and observing staining patterns, e.g. with immunofluorescence and automated image capture. In some embodiments, candidate agents can be screened in a model organism, e.g. zebrafish.

In some embodiments, the cell or organism can comprise a deleterious RNF216 mutation, and optionally, a deleterious OTUD4 mutation.

In some embodiments, the agonist of the RNF216/OTUD4 pathway can be identified as a therapeutic agent for a condition selected from the group consisting of: Alzheimer's; Parkinson's disease; cerebellar ataxia; neurodegenerative diseases; Gordon Holmes syndrome; and reproductive diseases involving hypothalamic and/or pituitary dysfunction. Examples of reproductive disease involving hypothalamic or pituitary can include, but are not limited to, hypogonadotropic hypogonadism; delayed puberty; amenorrhea; irregular menstrual function; polycystic ovary syndrome; erectile dysfunction; decreased libido; azoospermia; and infertility.

In some embodiments, a candidate agent which causes a statistically significant modulation selected from the group consisting of: decrease in the size of the optic tectum; decreased organization of axonal tract in the cerebellum; decrease in ophthalmic size; increase of acerebellar phenotypes; increased Nf-κB pathway activity; decreased GnRH secretion; and decreased gonadotrophin secretion; decreased ubiquitination activity or markers; modulation of the ataxia protein-protein interaction network; and decreased proteasome activity or markers; can be identified as an antagonist of the RNF216/OTUD4 pathway.

In some embodiments, the antagonist of the RNF216/OTUD4 pathway can be identified as a therapeutic agent for a condition selected from the group consisting of: precocious puberty; fibroids; endometriosis; polycystic ovary syndrome; sex-steroid-dependent cancer; breast cancer; and prostate cancer. In some embodiments, the antagonist of the RNF216/OTUD4 pathway can be identified as an agent for use in contraception or in vitro fertilization. In some embodiments, the antagonist of the RNF216/OTUD4 pathway can be identified as an agent that can induce a state of hypogonadotropic hypogonadism.

### Administration

The treatments described herein can comprise administering an effective dose of the treatment to the subject, e.g. to alleviate at least one symptom, e.g. of Gordon Holmes syndrome. As used herein, “alleviating a symptom” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the treatments described herein to subjects are known to those of skill in the art. Such methods can include, but are not limited to oral, parenteral, intravenous, intramuscular, subcutaneous, transdermal, airway (aerosol), cutaneous, injection or topical administration. Administration can be local or systemic.

The term “effective amount” as used herein refers to the amount of a treatment needed to alleviate at least one or more symptom of the disease or disorder, and relates to a sufficient amount of pharmacological composition to provide the desired effect. The term “therapeutically effective amount” therefore refers to an amount that is sufficient to effect a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for GnRH secretion, among others. The dosage can be determined by a physician and adjusted, as necessary, to suit observed effects of the treatment.

In certain embodiments, an effective dose of a treatment can be administered to a patient once. In certain embodiments, an effective dose of a treatment can be administered to a patient repeatedly. A treatment can be administered over a period of time, such as over a 5 minute, 10 minute, 15 minute, 20 minute, or 25 minute period. The administration can be repeated, for example, on a regular basis, such as hourly for 3 hours, 6 hours, 12 hours or longer or such as biweekly (i.e., every two weeks) for one month, two months, three months, four months or longer. In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The dosage ranges of a treatment, according to the methods described herein depend upon, for example, the form of the treatment, its potency, and the extent to which symptoms, markers, or indicators of a condition described herein are desired to be reduced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a treatment in, e.g. the treatment of a condition described herein, or to induce a response as described herein (e.g. GnRH secretion) can be determined by the skilled clinician. However, a treatment is considered “effective treatment,” as the term is used herein, if any one or all of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. ataxia); or (2) relieving the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of RNF216 mutations causing Gordon Holmes syndrome. When using an experimental animal model, efficacy of treatment is evidenced when a statistically significant change in a marker is observed.

In vitro and animal model assays are provided herein which allow the assessment of a given dose of an agent. By way of non-limiting example, the effects of a dose of an agent can be assessed by administration to a zebrafish as described elsewhere herein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%%, or more. As used herein, “reduction” or “inhibition” does not encompass a complete inhibition or reduction as compared to a reference level. “Complete inhibition” is a 100% inhibition as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., zebrafish, trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a condition, e.g. Gordon Holmes syndrome. A subject can be male or female.

A subject can be one who has been previously diagnosed with or identified as suffering from or having a condition in need of treatment (e.g. Gordon Holmes syndrome or neurologic degeneration or reproductive dysfunction) or one or more complications related to such a condition, and optionally, have already undergone treatment for the condition or the one or more complications. Alternatively, a subject can also be one who has not been previously diagnosed as having the condition or one or more complications related to the condition. For example, a subject can be one who exhibits one or more risk factors for a condition or one or more complications related to a condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In some embodiments, a RNF216 or OTUD4 polypeptide can be a variant of a sequence described herein, e.g. a variant of a RNF216 or UTUD4 polypeptide comprising the amino acid sequence of SEQ ID NO: 1 or SEQ ID NO: 5, or SEQ ID NO: 2. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, i.e., can ubiquitinate or deubiquitinate at at least 50% of the rate of the wildtype RNF216 or OTUD4, respectively. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or note, has more than 100% of the activity of wildtype RNF216 or OTUD4, e.g. 110%, 125%, 150%, 175%, 200%, 500%, 1000% or more.

One method of identifying amino acid residues which can be substituted is to align, for example, human RNF216 to a RNF216 homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Similarly, alignment with a related polypeptide from the same species, which does not show the same activity, can also provide guidance with respect to regions or structures required for activity. The variant amino acid or DNA sequence can be at least 90%, at least 95%, at least 96%, at least 97%, at least 98%, at least 99%, or more, identical to a native or reference sequence, e.g. SEQ ID NO: 1 or SEQ ID NO:2 or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired apoptotic activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, the RNF216 and/or OTUD4 polypeptide administered to a subject can comprise one or more amino acid substitutions or modifications. In some embodiments, the substitutions and/or modifications can prevent or reduce proteolytic degradation and/or prolong half-life of the polypeptide in the subject. In some embodiments, a RNF216 and/or OTUD4 polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “modulates the activity”, with respect to proteins and/or pathways, refers to downregulation (inhibits activity) or upregulation (activates or increases activity) of protein activity or function (e.g. inhibit activity of RNF216, or increase activity of RNF216). In one embodiment, the modulation occurs by directly inhibiting or increasing the activity of a protein, i.e. via direct physical interaction with the protein. In one embodiment, the activity of the protein is modulated indirectly, for example, in signaling, by inhibiting an upstream effector of the protein activity. In some embodiments, the activity of the protein is modulated by increasing or decreasing the level of the protein, e.g. by increasing or decreasing the expression of the gene encoding the protein. In some embodiments of this and other aspects of the invention, activity of the protein is inhibited or lowered by at least 5%, at least 10%, at least 20%, at least 30%, at least 40%, at least 50%, at least 60%, at least 70%, at least 80%, at least 90%, at least 95%, at least 98%, or 100% (e.g. complete loss of activity) relative to an uninhibited control. In some embodiments of this and other aspects of the invention, the inhibitor has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM. In some embodiments of this and other aspects of the invention, activity of the protein is increased by at least 5%, at least 10%, at least 20%, at least 30%, at least 40%, at least 50%, at least 60%, at least 70%, at least 80%, at least 90%, at least 1-fold, at least 1.1-fold, at least 1.5-fold, at least 2-fold, at least 3-fold, at least 4-fold, at least 5-fold, or more relative to an un-activated control, e.g. in absence of activating agent. In some embodiments of this and other aspects of the invention, the activator of protein activity has an EC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of diagnosing Gordon Holmes syndrome in a subject, the method comprising:
   detecting, in a sample obtained from a subject, the presence of a deleterious mutation in RNF216 and optionally, OTUD4;
   diagnosing the subject with Gordon Holmes syndrome.
2. The method of paragraph 1, wherein the subject is homozygous for the deleterious mutation.
3. The method of paragraph 1, wherein the deleterious mutation results in a truncation of the polypeptide as compared to SEQ ID NO: 1 or SEQ ID NO: 2.
4. The method of paragraph 1, wherein the detecting comprises detecting the size of the RNF216 and optionally, OTUD4 polypeptide.
5. The method of paragraph 1, wherein the deleterious mutation results in a polypeptide which differs by at least 10% from a polypeptide having the sequence of SEQ ID NO: 1 or SEQ ID NO: 2.
6. The method of paragraph 1, wherein the detecting comprises determining the amino acid sequence of the RNF216, and optionally, OTUD4 polypeptide.
7. The method of paragraph 1, wherein the detecting comprises determining the nucleic acid sequence of a nucleic acid molecule encoding RNF216 and optionally, OTUD4.
8. The method of any of paragraphs 1-7, wherein the deleterious mutation of RNF216 is selected from the group consisting of:
   c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and mutation of R717.
9. The method of paragraph 8, wherein the deleterious mutation of RNF216 is selected from the group consisting of:
   c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C.
10. The method of any of paragraphs 1-9, wherein the deleterious mutation of OTUD4 is selected from the group consisting of:
   998G>T and G333V.
11. A method of treating a subject, the method comprising:
   detecting, in a sample obtained from a subject, the presence of a deleterious mutation in RNF216 and optionally, OTUD4; and
   administering a treatment for Gordon Holmes syndrome to the subject.
12. The method of paragraph 11, wherein the treatment is increases the expression or activity of RNF216 and optionally, OTUD4.
13. The method of paragraph 11, wherein the treatment is selected from the group consisting of:
   an ubiquitin acting drug; a drug acting within the proteasome; a drug acting within the ataxia protein protein interaction network; a USP14 inhibitor; IU1; calcium channel blockers; pioglitazone; conenzyme Q10; idebenone; overexpression of heat shock proteins; inhibitors of ATXN1; zolpidem; varenicline; implantation of syngenic cerebellar, gonadotroph, or hypothalaus cells; and implantation of genetically-modified cerebellar, gonadotroph, or hypothalaus cells.
14. The method of paragraph 13, wherein the drug acting within the proteasome is a proteasome agonist selected from the group consisting of:
   SFN (1-isothio-cyanato-4(R)-methylsulfinylbutane); (methylsulfonyl)cyclohexylmethylisothiocyanante; oleuropein; betulinic acid; and derivatives thereof
15. An assay comprising;
   detecting, in a sample obtained from a subject, the presence of a deleterious mutation in RNF216 and optionally, OTUD4.
16. The assay of paragraph 15, wherein the detecting further comprises measuring whether the subject is homozygous or heterozygous for the deleterious mutation.
17. The assay of paragraph 15, wherein the deleterious mutation results in a truncation of the polypeptide as compared to SEQ ID NO: 1 or SEQ ID NO: 2.
18. The assay of paragraph 15, wherein the detecting comprises detecting the size of the RNF216 and optionally, OTUD4 polypeptide.
19. The assay of paragraph 15, wherein the deleterious mutation results in a polypeptide which differs by at least 10% from a polypeptide having the sequence of SEQ ID NO: 1 or SEQ ID NO: 2.
20. The assay of paragraph 15, wherein the detecting comprises determining the amino acid sequence of the RNF216, and optionally, OTUD4 polypeptide.
21. The assay of paragraph 15, wherein the detecting comprises determining the nucleic acid sequence of a nucleic acid molecule encoding RNF216 and optionally, OTUD4.
22. The assay of any of paragraphs 15-21, wherein the deleterious mutation of RNF216 is selected from the group consisting of:
   c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and mutation of R717.
23. The assay of paragraph 22, wherein the deleterious mutation of RNF216 is selected from the group consisting of:
   c.2251C>T; p.R751C; c.615_{—}616 delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C.
24. The assay of any of paragraphs 15-23, wherein the deleterious mutation of OTUD4 is selected from the group consisting of:
   998G>T and G333V.
25. A method of screening a candidate agent which modulates the RNF216/OTUD4 pathway, the method comprising;
   contacting a cell or organism with a candidate agent;
   measuring the modulation of a marker of the level of RNF216/OTUD4 pathway activity;
   wherein candidate agent is identified as an agent which modulates the RNF216/OTUD4 pathway when there is a statistically significant modulation of at least one marker.
26. The method of paragraph 25, wherein the marker of RNF216/OTUD4 pathway activity is selected from the group consisting of:
   size of the optic tectum; organization of axonal tracts in the cerebellum; ophthalmic size; the presence or absence of an acerebellar phenotype; Nf-κB pathway activity; GnRH secretion; gonadotrophin secretion; ubiquitination activity or markers; proteasome activity or markers, and the ataxia protein-protein interaction network.
27. The method of any of paragraphs 25-26, wherein a statistically significant modulation selected from the group consisting of:
   increase in the size of the optic tectum; increased organization of axonal tract in the cerebellum; increase in ophthalmic size; decrease of acerebellar phenotypes; decreased Nf-κB pathway activity; increased ubiquitination activity or markers;
   increased proteasome activity or markers; and modulation of the ataxia protein-protein interaction network. indicates the candidate agent is an agonist of the RNF216/OTUD4 pathway.
28. The method of any of paragraphs 25-27, wherein the cell or organism comprises a deleterious RNF216 mutation, and optionally, a deleterious OTUD4 mutation.
29. The method of any of paragraphs 25-28, wherein the agonist of the RNF216/OTUD4 pathway is identified as a therapeutic agent for a condition selected from the group consisting of:
   Alzheimer's; Parkinson's disease; ataxia; cerebellar ataxia; neurodegenerative diseases; Gordon Holmes syndrome; and reproductive diseases involving but not limited to hypothalamic and/or pituitary dysfunction.
30. The method of paragraph 29, wherein the reproductive disease involving hypothalamic or pituitary dysfunction is selected from the group consisting of:
   hypogonadotropic hypogonadism; delayed puberty; amenorrhea; irregular menstrual function; polycystic ovary syndrome; erectile dysfunction; decreased libido; azoospermia; and infertility.
31. The method of any of paragraphs 25-30, wherein a statistically significant modulation selected from the group consisting of:
   decrease in the size of the optic tectum; decreased organization of axonal tract in the cerebellum; decrease in ophthalmic size; increase of acerebellar phenotypes; increased Nf-κB pathway activity; decreased GnRH secretion; and decreased gonadotrophin secretion; decreased ubiquitination activity or markers; decreased proteasome activity or markers, and modulation of the ataxia protein-protein interaction network
indicates the candidate agent is an antagonist of the RNF216/OTUD4 pathway.
32. The method of paragraph 31, wherein the antagonist of the RNF216/OTUD4 pathway is identified as a therapeutic agent for a condition selected from the group consisting of:
   precocious puberty; fribroids; endometriosis; polycystic ovary syndrome; sex-steroid-dependent cancer; breast cancer; and prostate cancer.
33. The method of paragraph 31, wherein the antagonist of the RNF216/OTUD4 pathway is identified as an agent for use in contraception or in vitro fertilization.
34. The method of paragraph 31, wherein the antagonist of the RNF216/OTUD4 pathway is identified as an agent that can induce a state of hypogonadotropic hypogonadism.
35. The method of any of paragraphs 25-34, wherein the organism is a zebrafish.
36. A method of treating Gordon Holmes Syndrome in a subject in need thereof, the method comprising administering to the subject a proteasome agonist selected from the group consisting of:
   SFN (1-isothio-cyanato-4(R)-methylsulfinylbutane);
   (methylsulfonyl)cyclohexylmethylisothiocyanante; oleuropein; betulinic acid; and derivatives thereof
37. A method of treating or reducing cortical degeneration in a subject in need thereof, the method comprising administering to the subject a proteasome agonist selected from the group consisting of:
   SFN (1-isothio-cyanato-4(R)-methylsulfinylbutane); (methylsulfonyl)cyclohexylmethylisothiocyanante; oleuropein; betulinic acid; and derivatives thereof.

### EXAMPLES

### Example 1

The combination of ataxia and hypogonadism was first described over a century ago, but the genetic basis for this association has remained elusive. Whole-exome sequencing was performed on a patient with ataxia and hypogonadotropic hypogonadism, followed by targeted sequencing of candidate genes in other patients. Neurological and reproductive endocrine phenotypes were characterized in detail. The effects of sequence variants and epistatic interactions were quantitated in a zebrafish model.

Homozygous mutations in RNF216 and OTUD4, which encode an ubiquitin E3 ligase and a deubiquitinase, respectively, were found in three affected siblings within a consanguineous family. Additional screening identified compound heterozygous truncating mutations in RNF216 in an unrelated patient and single heterozygous mutations in four other patients. All patients had dementia. Neuronal loss was observed in cerebellar pathways and the hippocampus; surviving hippocampal neurons contained ubiquitin-immunoreactive intranuclear inclusions. Patients had defects at both hypothalamic and pituitary levels of the reproductive endocrine axis. Finally, knockdown of rnf216 or otud4 in zebrafish embryos resulted in defects in the eye, optic tectum, and cerebellum; combinatorial suppression of both genes exacerbated these phenotypes; and these defects could be rescued by wild-type but not mutant human RNF216 or OTUD4 mRNA.

As described herein, syndromic hypogonadotropic hypogonadism, ataxia and dementia is caused by inactivating mutations in RNF216 or the combination of mutations in RNF216 and OTUD4. These findings link disordered ubiquitination to neurodegeneration and reproductive dysfunction and highlight the utility of whole exome sequencing in combination with functional studies to unveil genetic interactions that cause disease.

### Introduction

Recent years have seen great advances in elucidating genetic causes of cerebellar ataxia, but a genetic defect was not previously found in about 40% of cases.¹ Among these are cases of ataxia occurring in association with failure of the reproductive axis, a syndrome first reported by Gordon Holmes² and which now bears his name (MIM 212840). Most patients with this syndrome are hypogonadotropic, with a defect in the production or release of gonadotropins by the pituitary gland,³⁻¹² although hypergonadotropic forms have been reported.⁹

Genes known to cause ataxia regulate a wide spectrum of cellular functions, including intracellular signaling, tau regulation, and mitochondrial function.¹ Strikingly, these genes have little functional overlap with genes associated with hypogonadotropic hypogonadism, which encode proteins involved in the normal development, migration, or mature function of the neurons that secrete gonadotropin-releasing hormone (GnRH).¹³

A decade ago, the inventors described the reproductive endocrine phenotype of a large consanguineous family manifesting cerebellar ataxia, dementia, and hypogonadotropic hypogonadism.¹² Described herein are mutations in ubiquitin-related genes in this kindred, as well as in unrelated patients, and demonstrations that these mutations are loss-of-function alleles that have an epistatic interaction.

### Methods

Subjects. Twelve subjects with ataxia and hypogonadotropic hypogonadism from eight families were referred to the Massachusetts General Hospital for clinical or genetic evaluation. This study was approved by the hospital's Human Research Committee, and all participants or an authorized representative provided written informed consent.

Genetic Analysis. DNA from Subject 3 underwent exome sequencing as detailed herein. Resulting candidate genes were sequenced in family members and in unrelated affected individuals. Several computer algorithms were used to predict the pathogenicity of variants and to identify interactions with known ataxia and hypogonadotropic hypogonadism genes.

Neuropathological Evaluation. After Subject 2's death, permission was obtained for an autopsy and the brain was collected within 6 h post mortem Immunohistochemistry was performed on tissue sections with antibodies against ubiquitin, tau, and α-synuclein. Electron microscopy was performed using standard procedures.

Endocrine Phenotyping. Subjects underwent reproductive endocrine phenotyping as described previously¹² and as described herein.

Zebrafish Investigation. All experiments were carried out with the approval of the Institutional Animal Care and Use Committee. Morpholino oligos (MO) for silencing of zebrafish rnf216 and otud4 were injected either alone or with wild-type (WT) or mutant human mRNA for RNF216, OTUD4, or both.

Identification and analysis of candidate genes and variants. Whole-exome sequencing for Subject 3 was performed through the Mendelian Sequencing Consortium at the Broad Institute. Exomic DNA was captured using the Agilent SureSelect Human All Exon v2 kit, and the captured libraries were sequenced using an Illumina HiSeq 2000 sequencer. The reads were mapped against the hg19 reference genome and variant calling was performed using the Broad pipeline. ³³⁻³⁴ Homozygous variants that were found in Subject 3, present at less than 1% frequency in the NHLBI GO Exome Sequencing Project,³⁵ and predicted to be damaging by PolyPhen2³⁶ were Sanger sequenced in the remaining siblings and one parent from the index pedigree. Variants that exhibited proper segregation were then sequenced in additional family members from the index pedigree as well as in another seven unrelated probands with Gordon Holmes syndrome and three affected family members of these probands. PolyPhen2,³⁶ SIFT,³⁷ PANTHER,³⁸ and Mutation Taster³⁹ were used to predict the pathogenicity of variants. DAPPLE,⁴⁰ GRAIL,⁴¹ Endeavour,⁴² InWeb scored network,⁴³ and CNVconnect⁴⁴ were used to identify potential interactions between RNF216 and other known ataxia and hypogonadotropic hypogonadism genes (Table 3).

Allele-specific PCR. Total RNA was extracted from lymphoblast cell lines for Subjects 5-7 and their parents using the RNeasy Kit (Qiagen). First-strand cDNA was synthesized with oligo-dT primers using the Superscript III First Strand Synthesis System kit (Invitrogen). Quantitative real-time polymerase chain reaction (PCR) was performed on an ABI 7000 Real Time PCR System. Primers are listed in Table 4. All reactions were performed in duplicate in separate wells, and resulting the delta Ct's were normalized to that of GAPDH.

Reproductive Endocrine Phenotyping. Subjects 2, 6, and 8 underwent blood sampling every 10-15 minutes for 3.25-12 h to examine the frequency, amplitude, and morphology of endogenous GnRH-induced luteinizing hormone (LH) pulse patterns. Pulses of LH secretion were identified using a modified version of the method of Santen and Bardin.⁴⁵⁻⁴⁶ Subjects 1, 2, 6, and 8 received exogenous, pulsatile GnRH with monitoring of gonadotropin responses as described previously.⁴⁷⁻⁴⁸ Hormone assays were performed as described in ref 48; the Second International Reference Preparation was used as the standard for LH and FSH.

Morpholino oligonucleotide design. Morpholino oligos were obtained from Gene Tools, LLC (Philomath, Oreg., USA). Reciprocal BLAST against the zebrafish genome identified a sole zebrafish ortholog of RNF126 (ENSDARG00000087893, 59% similarity, 46% identity). The antisense MO used for the silencing of zebrafish rnf216 targeted the intron 5′ donor splice site of the gene: CAGAAAATAGTTTGTGCTTACACAT (SEQ ID NO: 7).

Similar to rnf216, a sole ortholog of the human transcript for OTUD4 was identified in zebrafish (73% similarity, 61% identity). Although genomic annotation of the zebrafish locus indicated two possible splice isoforms (ENSDART00000149878 and ENSDART00000112598), RT-PCR in 3 days post fertilization (d.p.f.) embryos showed only one of these (ENSDART00000149878) to be expressed. The MO for otud4 targeted the donor splice site of intron 2: TACCCGAAGGAATTTGCGCACCATT (SEQ ID NO: 8).

Production of capped human mRNA. Wild-type RNF216 (NM_{—}207116.2) and OTUD4 (NM_{—}001102653.1) cDNAs were cloned into the pCR8 TOPO vector (Invitrogen). For production of capped human mRNA, the inserts were transferred to pCS2 using the Gateway technology (Invitrogen). The mutations identified in the index pedigree were introduced into RNF216 and OTUD4 by site-directed mutagenesis. All constructs were fully sequenced using exonic primers to verify the sequence.

Zebrafish embryo injections. Zebrafish were maintained and mated according to standard procedures.⁴⁹ Wild-type zebrafish embryos were injected at the 1-4 cell stage with 1 nl diluted MO and 0.5% phenol red. A dose of 6 ng of rnf216 or otud4-targeting MO was used as it gave the best rate of silencing and the minimum rate of mortality among the tested working dilutions (4-10 ng). For the epistatic interaction experiments, a cocktail containing 6 ng each of rnf216- and otud4-targeting MO was used.

For RNA rescue experiments, human wild-type or mutant RNF216 (NM_{—}207116.2) and OTUD4 (NM_{—}001102653.1) were cloned into the pCS2+ expression vector and transcribed in vitro using the SP6 Message Machine Kit (Ambion) to produce capped mRNA, and 200 pg of mRNA was injected for the rescue experiments.

Zebrafish embryo phenotyping. Injected embryos were scored at 3 days post fertilization for microphthalmia by measuring the area of the eye cup. For immunofluorescence studies, embryos were fixed in Dent's fixative (80% methanol, 20% dimethylsulfoxide) overnight at 4° C. After rehydration with decreasing series of methanol in PBS, the embryos were washed with PBS, permeabilized with 10 μg/ml proteinase K, and postfixed with 4% PFA. Embryos were then washed twice with IF buffer (0.1% Tween-20, 1% BSA in 1×PBS) for 10 min at room temperature. After incubation in blocking solution (10% FBS, 1% BSA in 1×PBS) for 1 h at room temperature, embryos were incubated with anti-α acetylated tubulin (1:1000; T7451, Sigma-Aldrich), overnight at 4° C. After two washes in IF buffer for 10 min, embryos were incubated with Alexa Fluor goat anti-mouse IgG (1:1000; A21207, A11001, Invitrogen) for 1 h at room temperature. Image acquisition and analysis was performed using Nikon NIS-Elements Advanced Research software. For each test, 50-100 embryos were injected and scored by two investigators who were blind to the injection cocktail. All experiments were performed 2-3 times and statistical significance was calculated using Student's t-test.

### Results

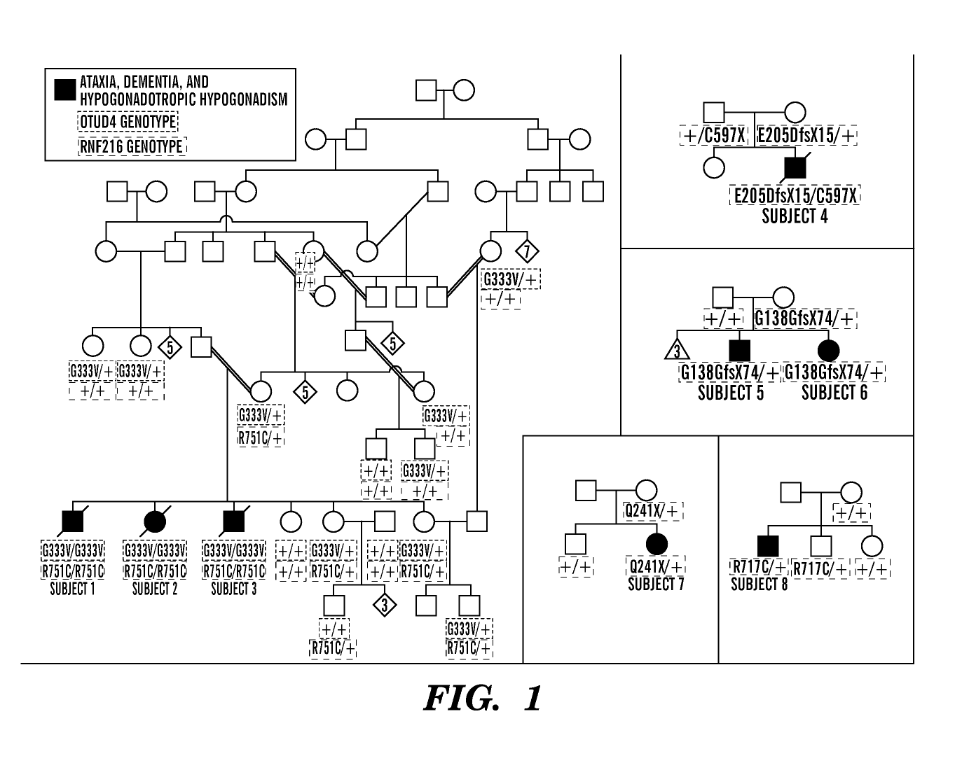

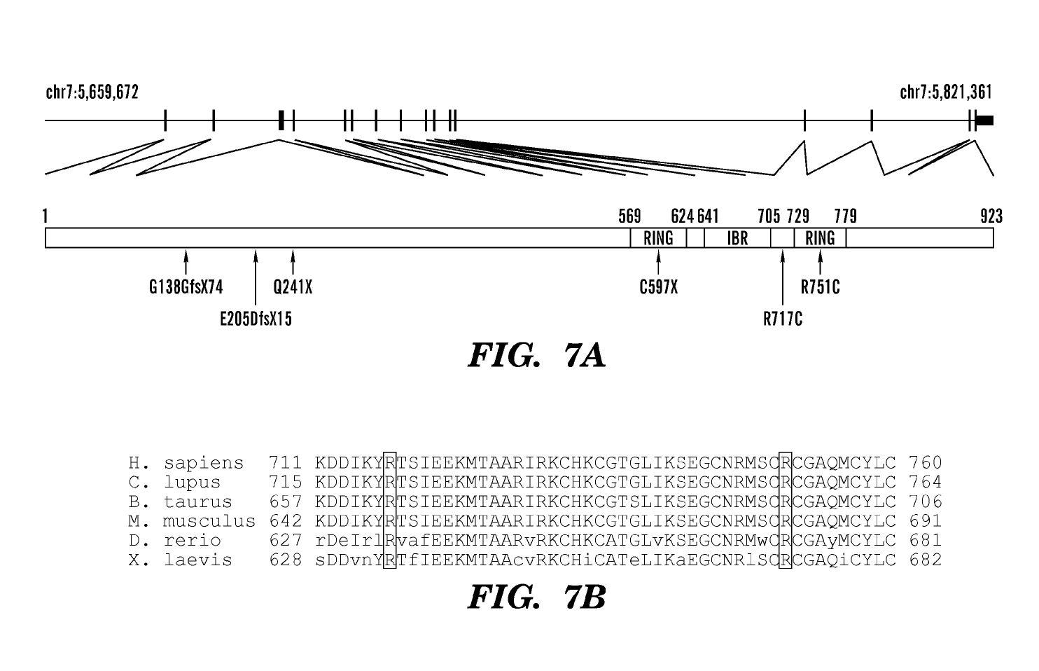

Genetic Studies. The index pedigree is a Palestinian family with first-cousin parents and six children, three of whom (Subjects 1-3) were affected by ataxia and hypogonadotropic hypogonadism. Whole-exome sequencing of Subject 3 identified thirteen variants that were homozygous, rare, and predicted to be deleterious (Table 2). Two of these thirteen variants were found to be homozygous in both affected siblings (Subjects 1 and 2) but not in any unaffected family members (FIG. 1): RNF216 (NM_{—}207111.3) c.2251C>T, p.R751C and OTUD4 (NM_{—}001102653.1) c.998G>T, p.G333V. RNF216 encodes an E3 ubiquitin-protein ligase. The R751 residue of RNF216 resides within the second RING finger domain of the protein and is conserved from zebrafish to humans (FIGS. 7A-7B). The R751C variant is predicted to be deleterious by four prediction programs and is absent from 13,006 control chromosomes in the NHLBI GO Exome Sequencing Project (ESP) [and 672 Middle Eastern chromosomes (including 36 Palestinian chromosomes)]. OTUD4 encodes a deubiquitinase. The altered G333V residue of OTUD4 was found in 2 of 13,006 chromosomes in the NHLBI ESP and in none of XXX Middle Eastern chromosomes. Although not as tightly conserved across species,¹⁴ the variant was predicted to be deleterious by three of four prediction programs.

These two genes were sequenced in nine additional unrelated affected individuals from seven families. No rare variants were identified in OTUD4, but deleterious mutations in RNF216 were identified in four probands (FIG. 1). Subject 4 had compound heterozygous mutations in RNF216 ([c.615_{—}616 delGA; p.E205DfsX15]+[c.1791T>A; p.C597X]). The nonsense change creates a premature stop codon in exon 11, and the frameshift mutation alters all amino acids C-terminal to residue 204, with premature termination after 15 amino acids (FIGS. 7A-7B). Three additional heterozygous mutations were identified: c.414delG; p.G138GfsX74 in Subjects 5 and 6 (siblings); c.721C>T; p.Q241X in Subject 7, and c.2149C>T; p.R717C in Subject 8. The R717 residue is highly conserved (FIGS. 7A-7B) and the R717C variant is predicted to be deleterious by all four prediction programs. No rare variants in RNF216 were found in Subjects 9-12.

None of these RNF216 variants were present in 13,006 chromosomes in the NHLBI Exome Sequencing Project, which did list five other heterozygous, overtly deleterious RNF216 variants (nonsense and splice-site) and nine likely deleterious variants (missense, <1% frequency, predicted to be pathogenic by four algorithms). Given this background level of genetic variation, the identification of deleterious mutations in 5 of 8 probands in this study exceeded what would be expected by chance (P<1×10⁻¹³, Fisher's exact test).

Grail, DAPPLE, Endeavour, InWeb scored network, and CNVconnect were used to identify connections between RNF216 and known ataxia and hypogonadotropic hypogonadism genes; no such connections were found.

Because only monoallelic changes in RNF216 were found in Subjects 5-8, allele-specific PCR was performed to determine whether the apparently wild-type allele harbored an occult mutation affecting mRNA transcription or stability. Robust expression of the wild-type alleles was seen (Figure SXXX), making the existence of such occult mutations unlikely.

Functional Testing of RNF216 and OTUD4 Variants

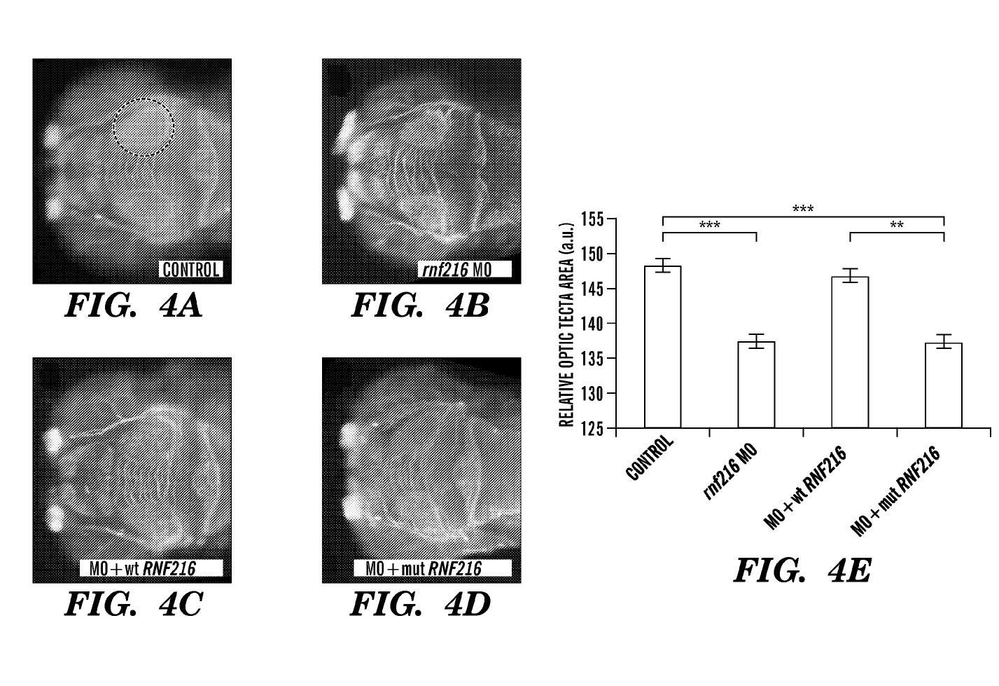

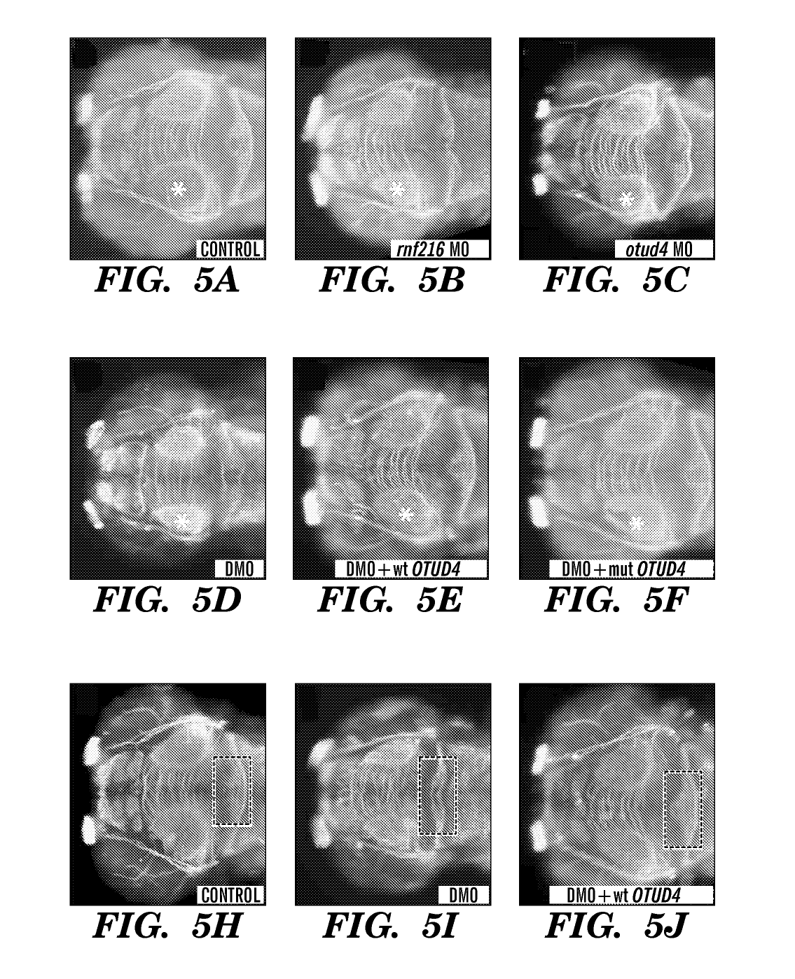

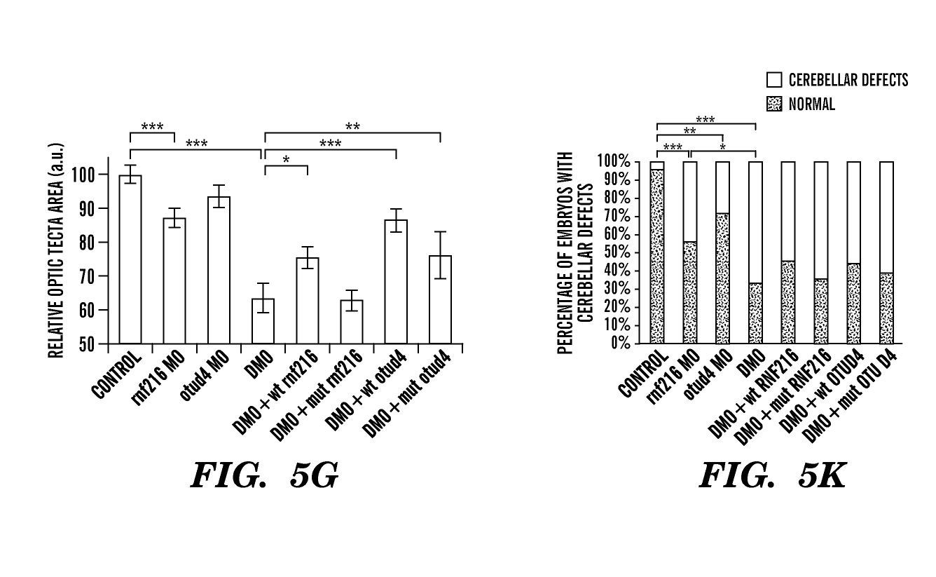

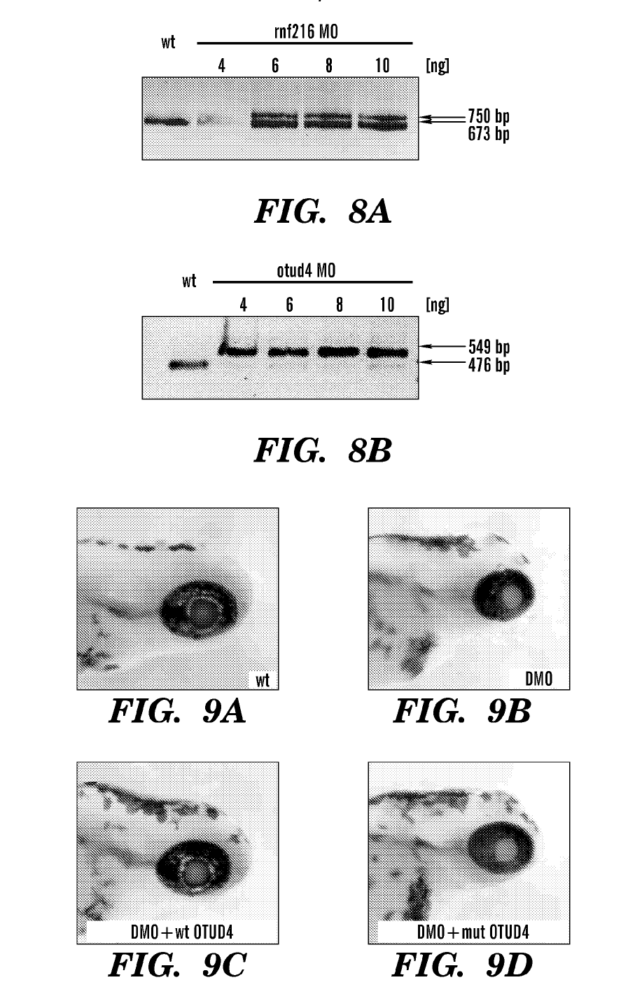

The possibility that RNF216 mutations might interact genetically with alleles at other loci was suggested by the finding in the index pedigree that the three affected subjects (and none of the unaffected family members) had homozygous mutations in OTUD4 that were rare, predicted to be pathogenic, and, intriguingly, also affected ubiquitination. To address this possibility, zebrafish development was utilized to test the functional consequences of the RNF216 and OTUD4 mutations from the index pedigree and to interrogate possible epistatic interactions.^{16, 17} Injection of a morpholino oligonucleotide (MO) that resulted in aberrant splicing of zebrafish rnf216 (FIGS. 8A-8B) caused a reduction in size of the optic tectum and disorganization of axonal tracts in the cerebellum (FIGS. 4, 5, S3). The tectal phenotype could be fully rescued by co-injection of human RNF216 mRNA, but injection of human RNF216 mRNA encoding the R751C variant failed to rescue the phenotype (FIGS. 4A-4E), suggesting that the mutation adversely impacts the encoded protein's function.

Similarly, injection of a splice-blocking MO against zebrafish otud4 (FIGS. 8A-8B), induced cerebellar defects and reduction in size of the optic tectum, although both phenotypes were milder than the ones observed for rnf216 MO alone (FIGS. 5A-5K).

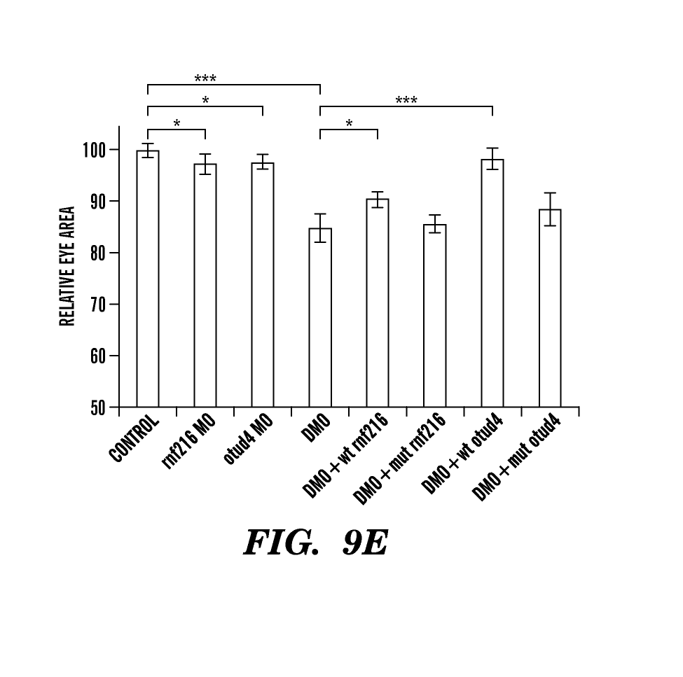

Combinatorial suppression of rnf216 and otud4 by co-injection of both rnf216 and otud4 MOs resulted in marked microphthalmia, compared to modest microphthalmia with either single MO injection (FIG. 9A-9E). Furthermore, double MOs induced a significant reduction in the size of the optic tecta compared to rnf216 MO alone (FIG. 5A-5K). Double MO injection also caused marked disorganization of the cerebellum in >60% of embryos, and a severe acerebellar phenotype was observed in ˜30% of double MO injected embryos compared to only 5-10% of embryos injected with either rnf216 or otuf4 MO (FIG. 5A-5K).

These phenotypes were specific, as co-injection of WT human RNF216 or OTUD4 mRNA rescued all phenotypes (FIGS. 5A-5K and 9A-9E). Critically, RNF216 mRNA encoding the R751C allele or OTUD4 mRNA encoding the G333V allele found in Subjects 1-3 were less effective than either WT mRNA in rescuing the phenotypes induced by double MO injection (FIGS. 5A-5K and 9A-9E), indicating that the mutant alleles encode functionally deficient proteins. These data support the possibility that epistatic interactions between loss-of-function mutations in RNF216 and OTUD4 contributed to the disease phenotype in the index pedigree.

Clinical Characteristics

Subjects 1-8, who carried variants in RNF216, shared similar clinical histories (Tables 1 and 5). They first presented with isolated hypogonadotropic hypogonadism, either with complete absence of pubertal development in adolescence or loss of reproductive endocrine function in early adulthood. They then developed dysarthria and/or ataxia in the third or fourth decade of life. Their ataxia was progressive and led to their becoming wheelchair-dependent and ultimately bedridden by the fourth or fifth decade of life. Dementia was also a prominent feature of Subjects 1-8, with personality changes and memory loss.

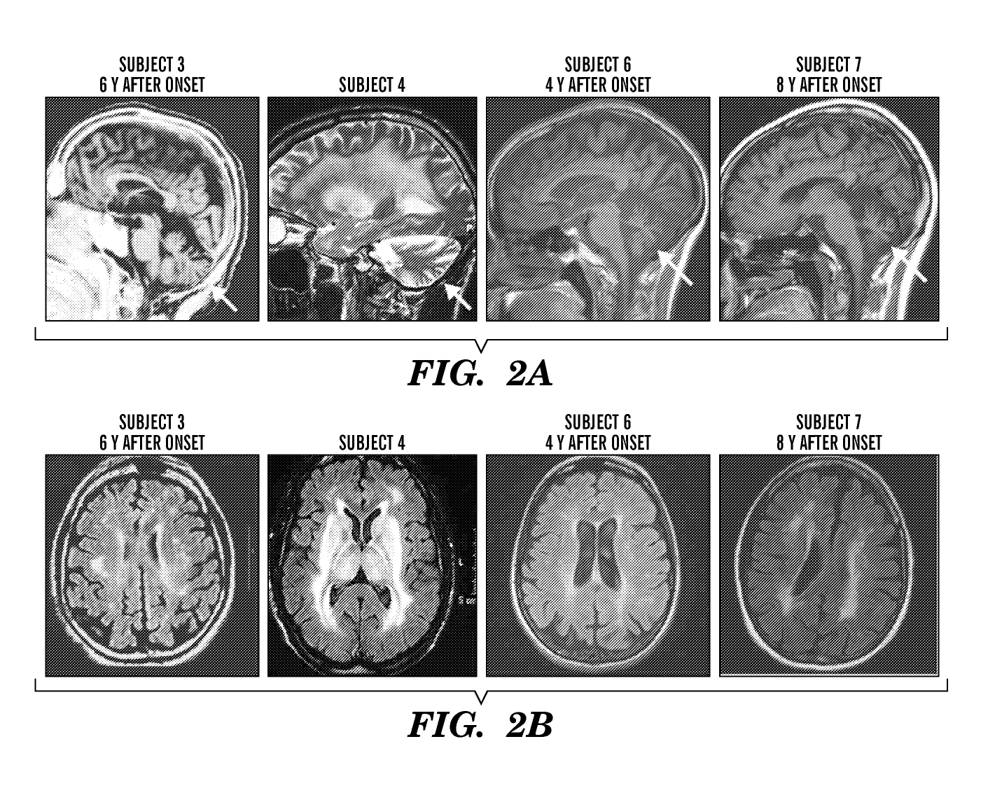

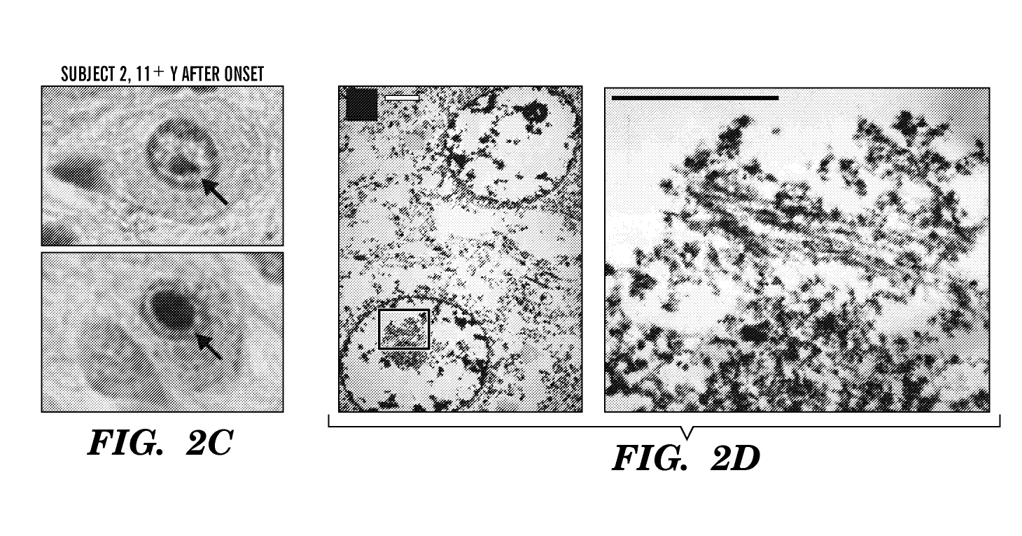

Neuroimaging of Subjects 1-8 revealed striking similarities, with cerebellar and cortical atrophy. There were patchy areas of hyperintensity in the subcortical white matter on T2-weighted imaging and fluid attenuated inversion recovery (FLAIR; Table 1, FIGS. 2A-2D). In Subject 7, these areas of hyperintensity were present at 18 y, ˜9 years prior to the onset of neurological symptoms; the cerebellum appeared normal at that time. No structural abnormalities of the pituitary gland were observed.

The clinical presentations of Subjects 9-12, who did not have variants in RNF216, were markedly different from those of Subjects 1-8 (Tables 1 and 5). Subjects 9-11 first presented in early childhood with ataxia that progressed slowly. Subjects 9-10 also had nystagmus, which was not observed in Subjects 1-8. Brain imaging of Subject 11 revealed cerebellar atrophy but not cortical atrophy. Subject 12 presented in late adolescence with personality changes, followed by tremor, dysarthria, and gait disturbance, and later ophthalmoplegia.

Extensive evaluation did not reveal any known causes of ataxia in any of the patients (Table 5), with the possible exceptions of Subjects 7 and 8, who had findings suggestive of mitochondrial abnormalities.

Neuropathologic Studies

At autopsy, the fixed brain of Subject 2 weighed 940 g (normal weight 1,300 g). The cerebellum was atrophic, with thin folia and gaping sulci, as were the inferior olives. Histopathology revealed selective neuronal loss and gliosis, with virtually complete loss of inferior olivary neurons and Purkinje cells in the cerebellum, although the pontine and cerebellar dentate nuclei and their outflow tracts were preserved. In the hippocampus, regions CA3 and CA4 were gliotic and depleted of neurons, whereas CA1 and CA2 were well preserved. Ubiquitin-immunoreactive nuclear inclusions were present in 1-5% of the pyramidal neurons in the hippocampal CA1 and CA2 regions (FIGS. 2A-2D) and in granule cell neurons in the dentate gyrus; these nuclear inclusions were not immunoreactive to antibodies against tau or α-synuclein (not shown). On electron microscopy, the intranuclear inclusions appeared as aggregates of fine filaments and granular material with no membrane separating them from the surrounding chromatin/nucleoplasm (FIGS. 2A-2D).

Reproductive Endocrine Studies

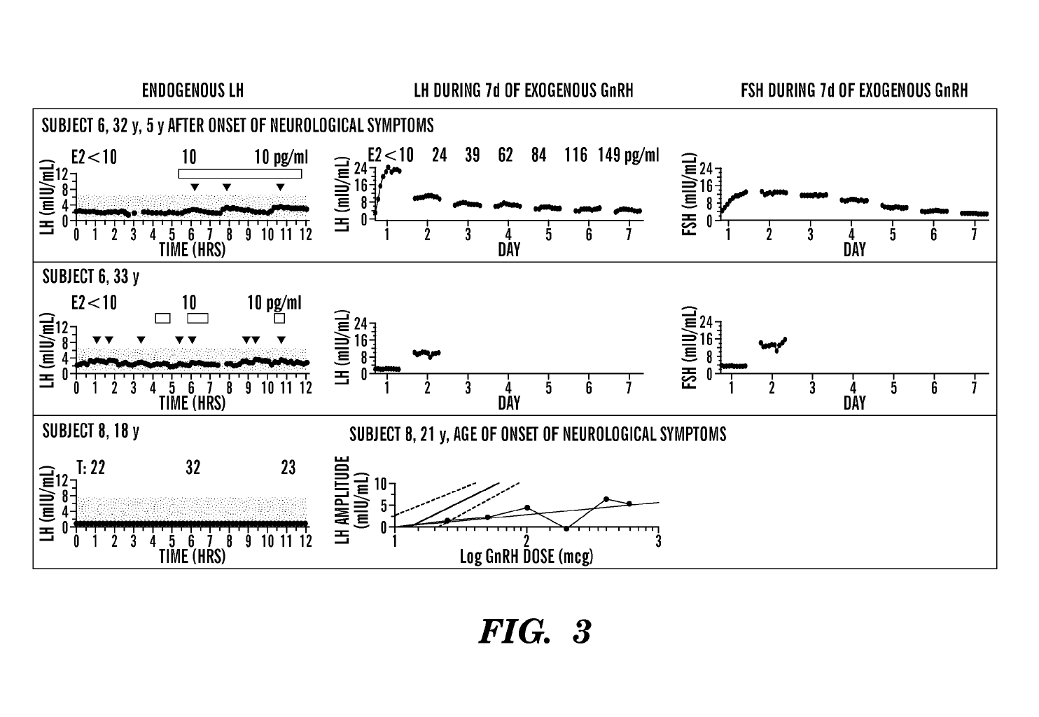

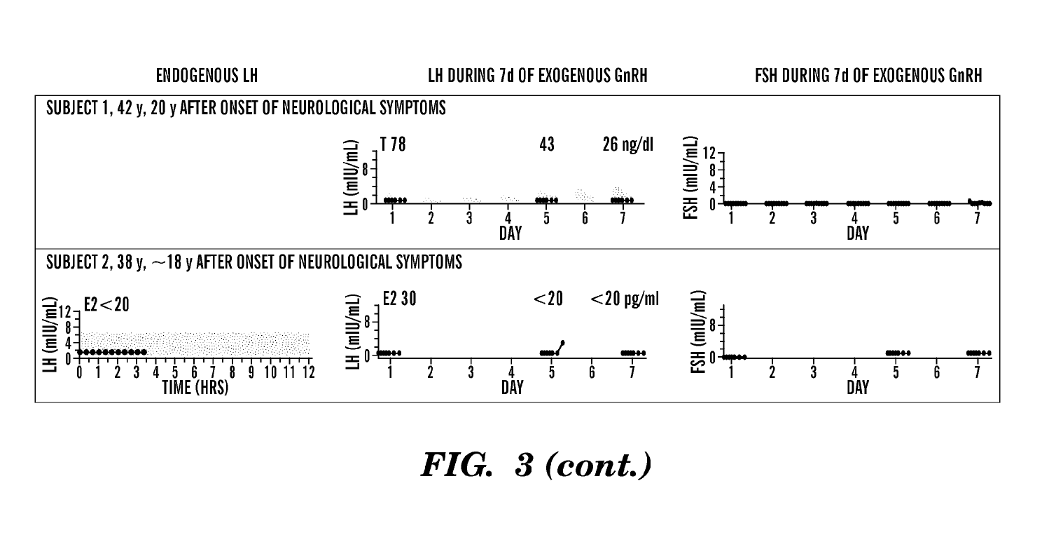

Subjects studied early in the course of their disease had findings consistent with GnRH deficiency that could be rescued with exogenous administration of GnRH. At 32 y, 5 y after developing secondary amenorrhea, Subject 6 demonstrated low-amplitude pulses of LH, indicating present but diminished GnRH secretion (FIG. 3). Exogenous GnRH at a standard dose induced an immediate robust increase in LH, consistent with stored pituitary hormone, and over the next 7 d pulsatile GnRH administration resulted in robust increases in gonadotropins and estradiol (FIG. 3) with growth of a dominant follicle observed by ultrasound (not shown). Though LH increased in response to GnRH, the usual morphology of LH pulses, with a sharp rise followed by a gradual decline,¹⁵ was not seen, suggesting a degree of pituitary dysfunction. Indeed, her pituitary responsiveness to GnRH waned over time: she was restudied 15 months after her initial study and found to have a lower response to GnRH on Day 1 of treatment, but a normal response on Day 2 (FIG. 3).

Subject 3 also demonstrated an intact but diminished pituitary response to GnRH at 32 y, 12 y after the onset of reproductive dysfunction and 3 y after the onset of dysarthria, with LH rising from 0.06 IU/L (baseline) to 5.8 IU/L (90 min after GnRH), and FSH rising from 0.14 IU/L (baseline) to 2.0 IU/L (120 min)¹²

Subject 8, studied at age 18 y, prior to onset of neurological symptoms, demonstrated an absence of endogenous pulsatile LH secretion (FIG. 3). His testicular volumes increased from 2 mL to 8 mL with exogenous pulsatile GnRH, with dose escalation from 25 to 600 ng/kg q2 h over 2 y, but did not increase further despite these very high doses. He had intact but markedly impaired pituitary responses to exogenous GnRH (FIG. 3), whereas direct gonadal stimulation with human chorionic gonadotropin treatment resulted in normalization of testosterone (459 ng/dL).

Pituitary responsiveness to GnRH was lost in subjects late in the course of their disease. Subjects 1 and 2), studied when they were bedridden, had LH at the limits of assay detection (FIG. 3 and ref 12) and failed to exhibit gonadotropin responses to pulsatile exogenous GnRH over 7 days (FIG. 3).

### Discussion

The biologic underpinnings for the association of ataxia with hypogonadotropic hypogonadism have eluded investigators for over a century. Described herein is the whole-exome sequencing of a single affected individual from a consanguineous family which led to the discovery of a recessive and oligogenic form of the disorder involving missense mutations in two genes, RNF216 and OTUD4. Both RNF216 and OTUD4 encode proteins that regulate ubiquitination, demonstrating that abnormalities in this fundamental cellular process can lead to pathology involving the cerebral white matter, the cerebellum, and the hypothalamic and pituitary components of the reproductive endocrine cascade.

The finding of compound heterozygous nonsense and frameshift mutations in RNF216 in Subject 4 firmly implicates RNF216 as a causative gene for this syndrome. However, heterozygous RNF216 mutations were found in Subjects 5-8. Allele-specific PCR revealed normal expression of the wild-type allele in these subjects, making it unlikely that these alleles harbored undetected mutations in regulatory regions affecting mRNA expression or other mutations that could affect mRNA stability. Moreover, haploinsufficiency of RNF216 is unlikely to be sufficient to cause disease, as several of the parents of Subjects 4-8 carried deleterious RNF216 mutations but were unaffected.

Oligogenicity offers a potential explanation for the apparently different inheritance patterns of RNF216. Oligogenic inheritance has been described in Bardet-Biedl syndrome, Bartter syndrome, Hirschsprung's disease, as well as isolated hypogonadotropic hypogonadism itself.^{16, 27-32} Under the oligogenic model, RNF216 mutations may act in conjunction with mutations at other genetic loci to cause disease.

Indeed, the phenotype in the index pedigree appears to have been caused by the interaction of hypomorphic mutations in both RNF216 and OTUD4 Inhibition of the function of either RNF216 or OTUD4 in a zebrafish model produced cerebellar hypoplasia, microphthalmia, and small optic tecta. The striking concordance of these phenotypes suggests that RNF216 and OTUD4 operate in the same pathway. This possibility is bolstered by the demonstration of epistatic interactions between RNF216 and OTUD4, with simultaneous knockdown of both genes resulting in markedly more severe phenotypes. Taken together, these findings support a digenic model in which the OTUD4 mutation plays an essential role in causing disease in the index pedigree. By extension, mutations in other as-yet unidentified loci may act in conjunction with the heterozygous deleterious mutations in RNF216 to cause disease in Subjects 5-8. Notably, in the absence of whole-exome data from the index pedigree, genetic analyses would like have been restricted to either RNF216 or OTUD4 alone, and discovery of mutations in one of these genes would have decreased the impetus to search for additional causative mutations. Thus, oligogenicity is likely to be increasingly recognized as methods capable of detecting this genetic architecture are more widely adopted.

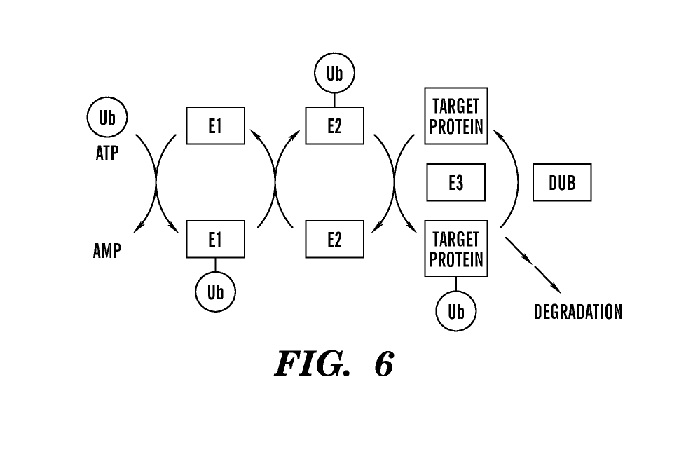

RNF216 encodes a E3 ubiquitin ligase that facilitates the attachment of ubiquitin to protein substrates and marks it for degradation by the proteasome (FIG. 6). Among known targets of RNF216 are upstream activators of NF-κB signaling.¹⁸⁻²⁰ Structural similarities exist between RNF216 and parkin, another E3 ubiquitin ligase which is mutated in an autosomal recessive form of Parkinson disease.²¹⁻²³ Parkin has wide-ranging neuroprotective effects, and mutations in parkin may interfere with the clearance of toxic proteins which then aggregate to form Lewy bodies, the histopathologic hallmark of Parkinson's disease.²¹⁻²³ The finding of neuronal intranuclear inclusions in Subject 2 may indicate similarities between RNF216-associated neurodegeneration and not only Parkinson's but also other neurodegenerative disorders in which protein aggregates are found, such as Huntington's and Alzheimer's disease.²⁴ The ubiquitin-immunoreactivity of these intranuclear inclusions further suggests a connection with the ubiquitin-ligase activity of RNF216. Finally, parkin has recently been shown to be important for mitophagy, impairment of which has been linked to neurodegeneration.²¹ Notably, Subjects 7 and 8 had evidence of abnormal mitochondrial function, but this was not a universal feature in patients with RNF216-associated neurodegeneration.

OTUD4 encodes a deubiquitinating enzyme (DUB) which allows target proteins and ubiquitin itself to be recycled. Deubiquitinases often function in partnership with specific E3 ligases. For example, the deubiquitinase ataxin-3 (so named because polyglutamine expansion in this protein leads to spinocerebellar ataxia 3) counteracts the ability of parkin to ubiquitinate itself.²⁵ Based on this and other examples,²⁶ it is possible that OTUD4 deubiquitination inhibits RNF216 itself, further linking these two proteins into a complex co-regulatory partnership.

The patients with RNF216 mutations share several clinical features that distinguish them from other patients with ataxia and hypogonadotropic hypogonadism, and indeed from patients with other ataxia syndromes in general. Unlike Gordon Holmes' original cases,² subjects with RNF216-associated neurodegeneration experienced not only ataxia but also progressive and ultimately debilitating dementia. Cerebral white-matter changes were observed on neuroimaging studies in all patients, suggesting that this is a defining feature of this syndrome. Furthermore, none of the patients had oculomotor abnormalities or retinopathy, while nystagmus and ophthalmoplegia were respectively seen in Subjects 9-10 and 12, who did not have RNF216 mutations.

The subjects with RNF216-associated neurodegeneration had dysfunction at multiple levels of the reproductive endocrine axis. Subjects 6 and 8 had demonstrable responses to extended treatment with exogenous GnRH, with recruitment of a dominant ovarian follicle in Subject 6 and testicular growth in Subject 8. This restoration of reproductive function with exogenous GnRH points to hypothalamic GnRH deficiency as the primary cause of their reproductive endocrine pathology. However, Subject 6's pituitary response was not fully normal, as she did not exhibit the typical robust pulses of gonadotropin secretion in response to GnRH, suggesting an additional element of pituitary dysfunction. Similarly, Subject 8 had markedly impaired responses to exogenous GnRH as evidenced by a right-shifted dose-response curve. The complete absence of responses to seven days of exogenous GnRH seen in Subjects 1 and 2, who underwent endocrine phenotyping late in the course of the disease, may represent progression of this pituitary dysfunction.

Neurodegenerative syndromes are largely defined by the cell types that are affected and the resulting pattern of symptoms. In this syndrome of ataxia, dementia, and reproductive endocrine dysfunction, loss of cerebellar, inferior olivary, and hippocampal neurons were directly observed on neuropathology. The neuroradiologic findings further demonstrate generalized changes in the cerebral white matter. The GnRH deficiency of Subjects 6 and 8 suggests loss of hypothalamic GnRH neurons (or other neurons that are essential for GnRH release), and the emergence of pituitary resistance to GnRH suggests eventual loss of the pituitary gonadotropes as well. Why certain cell types and not others are affected in the different neurodegenerative syndromes remains a fundamental unanswered question. The identification of a new neurodegenerative syndrome, as described herein, offers a novel line of investigation for understanding the basis for this selectivity.

In conclusion, it is demonstrated herein that loss-of-function mutations in RNF216 cause a distinct syndrome of ataxia, dementia, and hypogonadotropic hypogonadism. Further, provided herein is genetic and in vivo evidence to suggest that mutations in RNF216, an E3 ubiquitin ligase, and OTUD4, a deubiquitinase, can synergize to produce this syndrome, reinforcing the notion that mutational load within biological pathways can drive disease manifestation.¹⁷ Taken together, these data indicate a hitherto unknown role for the ubiquitination system in disorders of combined neurodegeneration and reproductive dysfunction, and demonstrate the power of whole-exome sequencing of single individuals, combined with in vivo functional studies, to identify disease-causing gene mutations and epistatic interactions.

The discovery described herein indicates dysregulation of NF-kB signaling, possibly via changes in ubiquitination, results from deleterious mutations of RNF216/OTUD4. The NF-kB pathway is involved in immunity, cancer, tumor survival, cell protection, cell proliferation, cellular stress, autophagy, and mitochondrial function. Germline mutations have been reported in a few other proteins that are components of the NF-kB pathway, but the phenotypes of those patients appear to be much more restricted to skin disease and recurrent infection. The phenotypes of the patients reported here are more broad and lethal. Additionally, RNF216 has homologies to parkin, which is mutated in some cases of autosomal recessive Parkinson's disease. The data indicates that there may links between NF-kB pathway and common neurodegenerative disorders, such as Parkinson's, as well as reproductive diseases. Thus, this discovery introduces new targets for the treatment of cerebellar ataxia, neurodegenerative syndromes, and reproductive diseases due to hypothalamic/pituitary dysfunction. The data described herein permit treatments which modulate the RNF216/OTUD4 pathway for all neurodegenerative diseases including Alzheimers, Parkinsons, as well as reproductive diseases affecting the hypothalamus or pituitary glands.

### REFERENCES

1. Sailer A, Houlden H. Recent advances in the genetics of cerebellar ataxias. Curr Neurol Neurosci Rep 2012; 12:227-36.
2. Holmes G. A form of familial degeneration of the cerebellum. Brain 1907; 30:466-89.
3. Volpé R, Metzler W S, Johnston M W. Familial hypogonadotropic eunuchoidism with cerebellar ataxia. J Clin Endocrinol Metab 1963; 23:107-15.
4. Lowenthal A, Bekaert J, Van Dessel F, van Hauwaert J. Familial cerebellar ataxia with hypogonadism. J Neurol 1979; 222:75-80.
5. Berciano J, Amado J A, Freijanes J, Rebollo M, Vaquero A. Familial cerebellar ataxia and hypogonadotropic hypogonadism: evidence for hypothalamic LHRH deficiency. J Neurol Neurosurg Psychiatry 1982; 45:747-51.
6. Fok A C, Wong M C, Cheah J S. Syndrome of cerebellar ataxia and hypogonadotropic hypogonadism: evidence for pituitary gonadotrophin deficiency. J Neurol Neurosurg Psychiatry 1989; 52:407-9.
7. Abs R, Van Vleymen E, Parizel P M, Van Acker K, Martin M, Martin J J. Congenital cerebellar hypoplasia and hypogonadotropic hypogonadism. J Neurol Sci 1990; 98:259-65.
8. De Michele G, Filla A, D'Armiento F P, et al. Cerebellar ataxia and hypogonadism. A clinicopathological report. Clin Neurol Neurosurg 1990; 92:67-70.
9. De Michele G, Filla A, Striano S, Rimoldi M, Campanella G. Heterogeneous findings in four cases of cerebellar ataxia associated with hypogonadism (Holmes' type ataxia). Clin Neurol Neurosurg 1993; 95:23-8.
10. Quinton R, Barnett P, Coskeran P, Bouloux P M. Gordon Holmes spinocerebellar ataxia: a gonadotrophin deficiency syndrome resistant to treatment with pulsatile gonadotrophin-releasing hormone. Clin Endocrinol (Oxf) 1999; 51:525-9.
11. Vielhaber S, Ebert A D, Feistner H, Herrmann M. Frontal-executive dysfunction in early onset cerebellar ataxia of Holmes' type. Clin Neurol Neurosurg 2000; 102:102-5.
12. Seminara S B, Acierno J S, Jr., Abdulwahid N A, Crowley W F, Jr., Margolin D H. Hypogonadotropic hypogonadism and cerebellar ataxia: detailed phenotypic characterization of a large, extended kindred. J Clin Endocrinol Metab 2002; 87:1607-12.
13. Balasubramanian R, Crowley W F, Jr. Isolated GnRH deficiency: a disease model serving as a unique prism into the systems biology of the GnRH neuronal network. Mol Cell Endocrinol 2011; 346:4-12.
14. HomoloGene: 35370.
15. Lavoie H B, Martin K A, Taylor E, Crowley W F, Hall J E. Exaggerated free alpha-subunit levels during pulsatile gonadotropin-releasing hormone replacement in women with idiopathic hypogonadotropic hypogonadism. J Clin Endocrinol Metab 1998; 83:241-7.
16. de Pontual L, Zaghloul N A, Thomas S, et al. Epistasis between RET and BBS mutations modulates enteric innervation and causes syndromic Hirschsprung disease. Proc Natl Acad Sci USA 2009; 106:13921-6.
17. Zaghloul N A, Katsanis N. Zebrafish assays of ciliopathies. Methods Cell Biol 2011; 105:257-72.
18. Chen D, Li X, Zhai Z, Shu H B. A novel zinc finger protein interacts with receptor-interacting protein (RIP) and inhibits tumor necrosis factor (TNF)- and IL1-induced NF-kappa B activation. J Biol Chem 2002; 277:15985-91.
19. Fearns C, Pan Q, Mathison J C, Chuang T H. Triad3A regulates ubiquitination and proteasomal degradation of RIP1 following disruption of Hsp90 binding. J Biol Chem 2006; 281:34592-600.
20. Miah S M, Purdy A K, Rodin N B, et al. Ubiquitylation of an internalized killer cell Ig-like receptor by Triad3A disrupts sustained NF-kappaB signaling. J Immunol 2011; 186:2959-69.
21. Pilsl A, Winklhofer K F. Parkin, P1NK1 and mitochondrial integrity: emerging concepts of mitochondrial dysfunction in Parkinson's disease. Acta Neuropathol 2011; 123:173-88.
22. Kumar P, Pradhan K, Karunya R, Ambasta R K, Querfurth H W. Cross-functional E3 ligases Parkin and C-terminus Hsp70-interacting protein in neurodegenerative disorders. J Neurochem 2011; 120:350-70.
23. Corti O, Lesage S, Brice A. What genetics tells us about the causes and mechanisms of Parkinson's disease. Physiol Rev 2011; 91:1161-218.
24. Saxena S, Caroni P. Selective neuronal vulnerability in neurodegenerative diseases: from stressor thresholds to degeneration. Neuron 2011; 71:35-48.
25. Durcan T M, Kontogiannea M, Bedard N, Wing S S, Fon E A. Ataxin-3 deubiquitination is coupled to Parkin ubiquitination via E2 ubiquitin-conjugating enzyme. J Biol Chem 2012; 287:531-41.
26. Nakada S, Tai I, Panier S, et al. Non-canonical inhibition of DNA damage-dependent ubiquitination by OTUB1. Nature 2010; 466:941-6.
27. Katsanis N, Ansley S J, Badano J L, et al. Triallelic inheritance in Bardet-Biedl syndrome, a Mendelian recessive disorder. Science 2001; 293:2256-9.
28. Katsanis N, Eichers E R, Ansley S J, et al. BBS4 is a minor contributor to Bardet-Biedl syndrome and may also participate in triallelic inheritance. Am J Hum Genet 2002; 71:22-9.
29. Badano J L, Leitch C C, Ansley S J, et al. Dissection of epistasis in oligogenic Bardet-Biedl syndrome. Nature 2006; 439:326-30.
30. Schlingmann K P, Konrad M, Jeck N, et al. Salt wasting and deafness resulting from mutations in two chloride channels. N Engl J Med 2004; 350:1314-9.
31. Nozu K, Inagaki T, Fu X J, et al. Molecular analysis of digenic inheritance in Bartter syndrome with sensorineural deafness. J Med Genet 2008; 45:182-6.
32. Sykiotis G P, Plummer L, Hughes V A, et al. Oligogenic basis of isolated gonadotropin-releasing hormone deficiency. Proc Natl Acad Sci USA 2010; 107:15140-4.
33. McKenna A, Hanna M, Banks E, et al. The Genome Analysis Toolkit: a MapReduce framework for analyzing next-generation DNA sequencing data. Genome Res 2010; 20:1297-303
34. DePristo M A, Banks E, Poplin R, et al. A framework for variation discovery and genotyping using next-generation DNA sequencing data. Nat Genet 2011; 43:491-8.
35. Exome Variant Server, NHLBI Exome Sequencing Project (ESP), Seattle, Wash. (URL: http://evs.gs.washington.edu/EVS/) [Jul. 6 2012].
36. Adzhubei I A, Schmidt S, Peshkin L, et al. A method and server for predicting damaging missense mutations. Nat Methods 2010; 7:248-9.
37. Kumar P, Henikoff S, Ng P C. Predicting the effects of coding non-synonymous variants on protein function using the SIFT algorithm. Nat Protoc 2009; 4:1073-81.
38. Mi H, Dong Q, Muruganujan A, Gaudet P, Lewis S, Thomas P D. PANTHER version 7: improved phylogenetic trees, orthologs and collaboration with the Gene Ontology Consortium. Nucleic Acids Res 2009; 38:D204-10.
39. Schwarz J M, Rodelsperger C, Schuelke M, Seelow D. MutationTaster evaluates disease-causing potential of sequence alterations. Nat Methods 2010; 7:575-6.
40. Rossin E J, Lage K, Raychaudhuri S, et al. Proteins encoded in genomic regions associated with immune-mediated disease physically interact and suggest underlying biology. PLoS Genet 2011; 7:e1001273.
41. Raychaudhuri S, Plenge R M, Rossin E J, et al. Identifying relationships among genomic disease regions: predicting genes at pathogenic SNP associations and rare deletions. PLoS Genet 2009; 5:e1000534.
42. Aerts S, Lambrechts D, Maity S, et al. Gene prioritization through genomic data fusion. Nat Biotechnol 2006; 24:537-44.
43. Lage K, Mollgard K, Greenway S, et al. Dissecting spatio-temporal protein networks driving human heart development and related disorders. Mol Syst Biol 2010; 6:381.
44. Longoni et al., in press.
45. Santen R J, Bardin C W. Episodic luteinizing hormone secretion in man. Pulse analysis, clinical interpretation, physiologic mechanisms. J Clin Invest 1973; 52:2617-28.
46. Hayes F J, McNicholl D J, Schoenfeld D, Marsh E E, Hall J E. Free alpha-subunit is superior to luteinizing hormone as a marker of gonadotropin-releasing hormone despite desensitization at fast pulse frequencies. J Clin Endocrinol Metab 1999; 84:1028-36.
47. Sprat D I, Finkelstein J S, Badger T M, Butler J P, Crowley W F, Jr. Bio- and immunoactive luteinizing hormone responses to low doses of gonadotropin-releasing hormone (GnRH): dose-response curves in GnRH-deficient men. J Clin Endocrinol Metab 1986; 63:143-50.
48. Seminara S B, Acierno J S, Jr., Abdulwahid N A, Crowley W F, Jr., Margolin D H. Hypogonadotropic hypogonadism and cerebellar ataxia: detailed phenotypic characterization of a large, extended kindred. J Clin Endocrinol Metab 2002; 87:1607-12.
49. Westerfield M. The zebrafish book: a guide for the laboratory use of zebrafish (Danio rerio). Eugene, Oreg.: University of Oregon Press; 2000.

TABLE 1
Clinical Phenotypes and RNF216 and OTUD4 Genotypes

| Family & | Subject | | Key Laboratory and | RNF216 | OTUD4 |
|---|---|---|---|---|---|
| Ethnicity | (Sex) | Key Clinical Features | Radiologic Findings | genotype | genotype |
| 1 | 1 (M) | No spontaneous puberty | 30 y, CT, prominent | [R751C] + | [G333V] + |
| Middle | | 22 y, onset of dysarthria | cerebellar and mild | [R751C] | [G333V] |
| Eastern | | 43 y, death (aspiration | cortical atrophy, | | |
| | | pneumonia) | hypodensities in cerebral | | |
| | | | white matter | | |
| | 2 (F) | 16 y, menarche, then | 30 y, CT, prominent | [R751C] + | [G333V] + |
| | | secondary amenorrhea | cerebellar and mild | [R751C] | [G333V] |
| | | 20 y, onset of personality | cortical atrophy, | | |
| | | changes | hypodensities in cerebral | | |
| | | 30 y, onset of dysarthria | white matter | | |
| | | 41 y, death (aspiration | | | |
| | | pneumonia) | | | |
| | 3 (M) | Normal puberty | 35 y, MRI, diffuse | [R751C] + | [G333V] + |
| | | 20 y, erectile dysfunction | parenchymal volume loss | [R751C] | [G333V] |
| | | 29 y, onset of dysarthria, | of cerebellum and | | |
| | | then progressive ataxia and | cerebral cortex, multiple | | |
| | | dementia | punctate and confluent | | |
| | | 47 y, death pulmonary | areas of T2 and FLAIR | | |
| | | embolism) | hyperintensity | | |
| 2 | 4 (M) | No spontaneous puberty | 23 y, MRI, cerebellar | [C597X] + | [=] + [=] |
| Caucasian | | 22 y, onset of dysarthria and | atrophy, widespread foci | [E205Dfs | |
| | | ataxia | of hyperintensity in | X15] | |
| | | 30 y, prominent chorea | cerebral white matter and | | |
| | | 36 y, death | thalami | | |
| 3 | 5 (M) | Normal puberty | 42 y, MRI, global atrophy, | [G138Gfs | [=] + [=] |
| Caucasian | | 36 y, hypogonadotropism, | diffusely scattered | X74] + | |
| | | onset of abnormal | periventricular foci of T2 | [=] | |
| | | movements, then | and FLAIR | | |
| | | progressive ataxia and | hyperintensity | | |
| | | dementia | | | |
| | 6 (F) | Normal puberty | 31 y, MRI, mild to | [G138Gfs | [=] + [=] |
| | | 27 y, oligo- then | moderate cerebellar | X74] + | |
| | | amenorrhea, onset of | atrophy, mild prominence | [=] | |
| | | memory problems | of ventricles and sulci, | | |
| | | 31 y, abnormal movements, | multiple small foci of T2 | | |
| | | then progressive ataxia and | hyperintensity | | |
| | | dementia | | | |
| 4 | 7 (F) | Primary amenorrhea | Abnormal mitochondria | [Q241X] + | [=] + [=] |
| Caucasian | | 27 y, onset of ataxia and | 18 y, MRI, normal | [=] | |
| | | dysarthria, then progressive | cerebellum, multiple T2 | | |
| | | ataxia and dementia | hyperintensities in | | |
| | | | subcortical while matter | | |
| | | | 35 y, MRI, marked | | |
| | | | cerebellar atrophy, | | |
| | | | increased number of foci | | |
| | | | of T2 hyperintensity | | |
| 5 | 8 (M) | No spontaneous puberty | Abnormal mitochondria | [R717C] + | [=] + [=] |
| Caucasian | | 21 y, slurred speech, | 17 y, slight prominence of | [=] | |
| | | imbalance, then progressive | fissures in cerebellum | | |
| | | ataxia, mood changes, and | 23 y, severe cerebellar and | | |
| | | memory impairment | mild cerebral atrophy, | | |
| | | | multiple foci of T2 and | | |
| | | | FLAIR hyperintensity | | |
| 6 | 9 (M) | No spontaneous puberty | | [=] + [=] | [=] + [=] |
| Caucasian | | 5 y, onset of ataxia, | | | |
| | | nystagmus | | | |
| | | 34 y, still able to walk | | | |
| | 10 (M) | No spontaneous puberty | | [=] + [=] | [=] + [=] |
| | | 5 y, onset of ataxia, | | | |
| | | nystagmus | | | |
| | | 32 y, still able to walk | | | |
| 7 | 11 (M) | No spontaneous puberty | 4 y, normal cerebellum | [=] + [=] | [=] + [=] |
| Caucasian | | 4 y, onset of ataxia | 17 y, cerebellopontine | | |
| | | 28 y, ataxia stable | atrophy, global white- | | |
| | | | matter abnormality in | | |
| | | | basal ganglia and cortex | | |
| 8 | 12 (F) | Normal puberty | 21 y, mild atrophy, T2 and | [=] + [=] | [=] + [=] |
| Asian | | 17 y, onset of behavior | FLAIR hyperintensity in | | |
| | | problems, then tremor, | brainstem, thalami, | | |
| | | dysarthria, ataxia | internal capsules, insulae, | | |
| | | 19 y, internuclear | basal ganglia, and | | |
| | | ophthalmoplegia | periventricular regions | | |
| | | 24 y, onset of | | | |
| | | hypogonadotropism | | | |

TABLE 2
Homozygous variants found in Subject 3 and predicted to be deleterious

| | Gene | | | Amino-acid | Segregation |
|---|---|---|---|---|---|
| Chromosome | name | RefSeq Accession | Nucleotide change | change | with disease |
| 1 | AGRN | NM_198576.2 | c.1123G > T | p.A375S | N |
| 1 | DVL1 | NM_004421.2 | c.997C > T | p.L333F | N |
| 1 | FNDC7 | NM_001144937.1 | c.1100A > G | p.N367S | N |
| 1 | GABPB2 | NM_144618.2 | c.439A > G | p.K147E | N |
| 1 | LCE2A | NM_178428.3 | c.59G > A | p.C20Y | N |
| 1 | OR6K2 | NM_001005279.1 | c.236C > T | p.P79L | N |
| 2 | PRPF40A | NM_017892.3 | c.1480-8_1480-3 | splice | N |
| | | | delTGCTAT | | |
| 2 | TTC21B | NM_024753.3 | c.3004C > G | p.L1002V | N |
| 2 | FARP2 | NM_014808.2 | c.2185C > T | p.R729W | N |
| 4 | OTUD4 | NM_001102653.1 | c.998G > T | p.G333V | Y |
| 7 | RNF216 | NM_207111.3 | c.2251C > T | p.R751C | Y |
| 10 | LOXL4 | NM_032211 | c.1135C > T | p.R379C | N |
| 16 | HYDIN | NM_032821.2 | c.12896G > C | p.C4299S | N |

TABLE 3
Known idiopathic hypogonadotropic hypogonadism (IHH) and
ataxia genes used for DAPPLE, GRAIL, Endeavour, Inweb scored
network, and CNVconnect analyses. Genes for which the genetic
mutation is expansion of triplet repeats were not included
in analysis, as the role of endogenous gene function in
pathogenesis in these disorders is unclear.

| | Ataxia | Ataxia | Ataxia | Ataxia |
|---|---|---|---|---|
| IHH | (AR) | (AD) | (X-linked) | (mito) |
| KAL1 | FXN | SPTBN2 | ABC7 | TK2 |
| FGF8 | POLG | ATXN10 | | ATP6 |
| FGFR1 | TTPA | TTBK2 | | |
| HS6ST1 | MTTP | KCNC3 | | |
| CHD7 | ATM | PRKCG | | |
| NELF | APTX | ITPR1 | | |
| PROK2 | SETX | IFRD1 | | |
| PROKR2 | HEXA | PDYN | | |
| TAC3 | HEXB | FGF14 | | |
| TACR3 | PMM2 | AFG3L2 | | |
| LEP | SACS | BEAN1 | | |
| LEPR | PhyH | TGM6 | | |
| KISS1 | PEX7 | NOP56 | | |
| KISS1R | CYP27A1 | KCNA1 | | |
| GNRH1 | SYNE1 | CACNA1A | | |
| GNRHR | ADCK3 | CACNB4 | | |
| | NPC1 | SLC1A3 | | |
| | NPC2 | SAX1 | | |

TABLE 4
Primers for allele-specific PCR.

RNF216 c.414delG, p.G138GfsX74 (Subjects 5-6 and family members)

| | |
|---|---|
| Forward | 5′-GGATGACTACGGTGAATTTCTGG-3′ (SEQ ID NO: 9) |
| Reverse | 5′-CACTTGGCTTAGTGAATTCAGAGATT-3′ (SEQ ID NO: 10) |
| Probe (wild-type) | 5′-FAM-TCTTGGCCCTCCTGG-MGB-3′ (SEQ ID NO: 11) |
| Probe (mutant) | 5′-TCTTGGCCTCCTGG-3′ (SEQ ID NO: 12) |

RNF216 c.721C>T, p.Q241X (Subject 7 and family members)

| | |
|---|---|
| Forward | 5′-TGGTTAGATCATCCTTACTTCCAGTC-3′ (SEQ ID NO: 13) |
| Reverse | 5′-GAGGAACGACCTGGTTTGTTATT-3′ (SEQ ID NO: 14) |
| Probe (wild-type) | 5′-FAM-TGAACCAACAGCCCC-MGB-3′ (SEQ ID NO: 15) |
| Probe (mutant) | 5′-FAM-TGAACCAATAGCCCC-MGB-3′ (SEQ ID NO: 16) |

TABLE 5
Case details.

| Subject- | Reproductive | Neurological | Brain Imaging | Laboratory and | RNF216 | OTUD4 |
|---|---|---|---|---|---|---|
| Sex | History | History | Studies | Genetic Studies | Genotype | Genotype |
| 1-M | 16 y, | 22 y, | 30 y, CT, | Negative genetic | [R751C] + | [G333V] + |
| (brother | evaluated | dysarthria | prominent | testing for SCA17 | [R751C] | [G333V] |
| of | for delayed | 24 y, ataxia | cerebellar and | | | |
| Subjects | puberty | 30 y, | mild cortical | | | |
| 2-3) | 42 y, no | wheelchair | atrophy, | | | |
| | response to | bound | hypodensities | | | |
| | exogenous | 43 y, death | in cerebral | | | |
| | GnRH | from | white matter | | | |
| | | aspiration | | | | |
| | | pneumonia | | | | |
| 2-F | 16 y, | early 20's, | 30 y, CT, | | [R751C] + | [G333V] + |
| (sister | menarche, | personality | prominent | | [R751C] | [G333V] |
| of | two | changes | cerebellar and | | | |
| Subjects | menstrual | 30 y, | mild cortical | | | |
| 1, 3) | periods then | dysarthria | atrophy, | | | |
| | amenorrhea | early 30's, | hypodensities | | | |
| | 38 y, no | ataxia | in cerebral | | | |
| | response to | 36 y, | white matter | | | |
| | exogenous | wheelchair | | | | |
| | GnRH | bound | | | | |
| | | 41 y, death | | | | |
| | | from | | | | |
| | | aspiration | | | | |
| | | pneumonia | | | | |
| 3-M | Normal | 29 y, mild | 35 y, MRI, | Notable for type IIB | [R751C] + | [G333V] + |
| (brother | puberty | dysarthria | diffuse | hyperlipidemia | [R751C] | [G333V] |
| of | 20 y, erectile | next 18 y, | parenchymal | Negative laboratory | | |
| Subjects | dysfunction | progressive | volume loss of | testing for disorders | | |
| 1-2) | | ataxia and | cerebellum and | of glycosylation, | | |
| | | dementia | cerebral | adrenoleukodystrophy, | | |
| | | 47 y, death | cortex, | metachromatic | | |
| | | possibly | multiple | leukodystrophy, | | |
| | | from | punctate and | Krabbe and Refsum | | |
| | | pulmonary | confluent areas | diseases, multiple | | |
| | | embolism | of T2 and | sclerosis, amino acid | | |
| | | | FLAIR | abnormality, vitamin | | |
| | | | hyperintensity | E deficiency | | |
| | | | | Normal karyotype. | | |
| | | | | Negative genetic | | |
| | | | | testing for HD, | | |
| | | | | DRPLA, FRDA, | | |
| | | | | FXTAS, SBMA, | | |
| | | | | SCA 1-3, 6-8, 10, 12 | | |
| 4-M | 19 y, | 22 y, gradual | 23 y, cerebellar | Negative for | [C597X] + | [=] + [=] |
| | evaluated | onset of | atrophy, | mitochondrial | [E205DfsX15] | |
| | for absent | dysarthria | widespread | disorders, vitamin E | | |
| | puberty | and gait | foci of | deficiency, Wilson's | | |
| | | ataxia | hyperintensity | disease, multiple | | |
| | | 23 y, | in cerebral | sclerosis, and | | |
| | | dysarthria, | white matter | lysosomal storage | | |
| | | mild ataxia | and thalami. | diseases. | | |
| | | and | | Negative genetic | | |
| | | dysmetria | | testing fror SCA | | |
| | | 30 y, normal | | 1, 2, 3, 6, 7, DRPLA, | | |
| | | eye | | and HD. | | |
| | | movements, | | | | |
| | | prominent | | | | |
| | | chorea | | | | |
| | | 34 y, unable to | | | | |
| | | speak | | | | |
| | | 36 y, death | | | | |
| 5-M | Normal | 36 y, | 42 y, MRI, | Negative laboratory | [G138GfsX74] + | [=] + [=] |
| (brother | puberty, | abnormal | global atrophy, | testing for lysosomal | [=] | |
| of | 36 y, | movements | diffusely | enzyme deficiency, | | |
| Subject | testosterone | next 6 y, | scattered | creatine transporter | | |
| 6) | deficiency | progressive | periventricular | deficiency, deficiency | | |
| | | ataxia and | foci of T2 and | of folate, vitamin B12 | | |
| | | dementia | FLAIR | or E, Tay-Sachs or | | |
| | | | hyperintensity | Wilson's diseases, | | |
| | | | | homocystinuria, fatty | | |
| | | | | acid oxidation | | |
| | | | | disorder, amino or | | |
| | | | | organic acid | | |
| | | | | abnormality, | | |
| | | | | metachromatic | | |
| | | | | leukodystrophy, | | |
| | | | | rheumatologic | | |
| | | | | disorder, multiple | | |
| | | | | sclerosis, arsenic, | | |
| | | | | lead, copper, or | | |
| | | | | mercury poisoning | | |
| | | | | Negative genetic | | |
| | | | | testing for DRPLA, | | |
| | | | | HDL2, MELAS, | | |
| | | | | MERRF, NARP, | | |
| | | | | SCA2, 3, and 17, | | |
| | | | | normal chromosomal | | |
| | | | | microarray | | |
| 6-F | Normal | 27 y, problems | 31 y, MRI, mild | Notable for | [G138GfsX74] + | [=] + [=] |
| (sister | puberty | with memory | to moderate | normocytic anemia | [=] | |
| of | 13 y, | 31 y, | cerebellar | and positive ANA | | |
| Subject | menarche | abnormal | atrophy, mild | (1:160) with | | |
| 5) | and regular | movements | prominence of | homogenous pattern | | |
| | menses | next 4 y, | ventricles and | Negative laboratory | | |
| | 20-27 y, oral | progressive | sulci, multiple | testing for celiac, | | |
| | contraceptive | ataxia and | small foci of | Lyme, Niemann-Pick | | |
| | pills | dementia | T2 | type C, Tay-Sachs, | | |
| | 27 y, stopped | | hyperintensity | and Wilson's | | |
| | oral | | | diseases, syphilis, | | |
| | contraceptives, | | | acanthocytosis, | | |
| | oligo- | | | mitochondrial | | |
| | then | | | disorders, amino or | | |
| | amenorrhea | | | organic acidemias, | | |
| | 32 y and 33 y, | | | adrenal | | |
| | low- | | | leukodystrophy, | | |
| | amplitude | | | neuronal ceroid | | |
| | LH pulses, | | | lipofuscinosis, | | |
| | responsive | | | lysosomal enzyme | | |
| | to | | | deficiency, | | |
| | exogenous | | | lipoprotein | | |
| | GnRH | | | abnormality, | | |
| | | | | deficiency of | | |
| | | | | coenzyme Q, folate, | | |
| | | | | vitamins B12, E, or | | |
| | | | | coenzyme Q | | |
| | | | | Negative genetic | | |
| | | | | testing for AOA1-2, | | |
| | | | | AVED, DRPLA, | | |
| | | | | FRDA, HD, MELAS, | | |
| | | | | MERRF, MSS, | | |
| | | | | NARP, SANDO, | | |
| | | | | SCA1-3, 6-8, 10, 14, | | |
| | | | | and 17 | | |
| 7-F | Primary | 27 y, ataxia | 18 y, MRI, | Notable for abnormal | [Q241X] + [=] | [=] + [=] |
| | amenorrhea | and | normal | and branched | | |
| | 19-24 y, | dysarthria | cerebellum, | mitochondria on skin | | |
| | regular | next 9 y, | multiple 1-2 | biopsy, elevated | | |
| | menses | progressive | mm foci of | pyruvate/lactate ratio, | | |
| | with | ataxia and | T2 | and mitochondrial | | |
| | estradiol/ | dementia | hyperintensity | enzyme studies | | |
| | medroxy- | | in subcortical | suggestive of a | | |
| | progesterone | | white matter of | complex I disorder; | | |
| | 24 y, | | bilateral | and a positive ANA | | |
| | amenorrhea | | frontal lobes. | but absence of other | | |
| | after | | 35 y, MRI, | autoantibodies | | |
| | stopping | | marked | Negative laboratory | | |
| | treatment | | cerebellar | testing for celiac, | | |
| | | | atrophy, | Lyme, Wilson's, | | |
| | | | increased | Whipple, and Tay- | | |
| | | | number of foci | Sachs diseases, | | |
| | | | of T2 | hemochromatosis, | | |
| | | | hyperintensity, | syphilis, deficiency of | | |
| | | | inverted lactate | vitamins B12 or E, | | |
| | | | peak on | amino acid | | |
| | | | spectroscopy | abnormality, multiple | | |
| | | | | sclerosis | | |
| | | | | Negative genetic | | |
| | | | | testing for SCA 1-8, | | |
| | | | | 10, 12-14, 17, 28, | | |
| | | | | FRDA, DRPLA, | | |
| | | | | AOA1-2, AVED, | | |
| | | | | MELAS, MERF, | | |
| | | | | MSS, NARP | | |
| 8-M | 17 y, | 21 y, slurred | 17 y, very slight | Notable for | [R717C] + [=] | [=] + [=] |
| | evaluated | speech and | prominence of | subsarcolemmal | | |
| | for lack of | poor balance | fissures in | accumulation of | | |
| | pubertal | 22 y, difficulty | cerebellum | mitochondria with | | |
| | development | keeping | 21 y, prominent | complex cristae and | | |
| | 18 y, absence | track of a | cerebellar | abnormally elongated | | |
| | of pulsatile | conversation | atrophy, no | forms on muscle | | |
| | LH | 23 y, issues | supratentorial | biopsy, mitochondrial | | |
| | secretion | with anger, | abnormalities | enzyme studies | | |
| | 19-21 y, | depression, | 23 y, severe | suggestive of | | |
| | partial | and temper | cerebellar | complex I-IV | | |
| | response to | control | atrophy, mild | disorder and | | |
| | exogenous | next 3 y, | cerebral | decreased citrate | | |
| | pulsatile | progressive | atrophy, | synthase activity | | |
| | GnRH | ataxia and | multiple foci | Negative for | | |
| | | executive | of T2 and | sarcoidosis, Wilson's, | | |
| | | memory | FLAIR | and celiac diseases, | | |
| | | impairment | hyperintensity | deficiency of vitamin | | |
| | | | in subcortical | E, | | |
| | | | and | adrenoleukodystrophy, | | |
| | | | periventricular | autoimmune, | | |
| | | | white matter, | peroxisomal, thyroid, | | |
| | | | no lactate peak | liver, renal | | |
| | | | | abnormalities | | |
| | | | | Normal karyotype; | | |
| | | | | negative genetic | | |
| | | | | testing for SCA 1-3, | | |
| | | | | 5-8, 10, 14, 17, | | |
| | | | | FRDA, DRPLA, | | |
| | | | | AOA1-2 | | |
| 9-M | 14 y, | 5 y, | | | [=] + [=] | [=] + [=] |
| | evaluated | olivocerebellar | | | | |
| | for lack of | pontine | | | | |
| | puberty | ataxia | | | | |
| | | diagnosed, | | | | |
| | | nystagmus | | | | |
| | | 34 y, still able | | | | |
| | | to walk | | | | |
| 10-M | 12 y, | 5 y, ataxia | | | [=] + [=] | [=] + [=] |
| (brother | evaluated | diagnosed, | | | | |
| of | for | nystagmus | | | | |
| Subject | hypogonado- | 32 y, still able | | | | |
| 9) | tropic | to walk | | | | |
| | hypogonadism | | | | | |
| 11-M | 20 y, | 4 y, ataxia | 4 y, normal | Normal karyotype | [=] + [=] | [=] + [=] |
| | evaluated | 28 y, ataxia | cerebellum | | | |
| | for lack of | stable | 10 y, marked | | | |
| | puberty | | cerebellar | | | |
| | | | atrophy | | | |
| | | | 16 y, further | | | |
| | | | cerebellar | | | |
| | | | atrophy | | | |
| | | | 17 y, | | | |
| | | | cerebellopontine | | | |
| | | | atrophy, no | | | |
| | | | cortical | | | |
| | | | atrophy, global | | | |
| | | | white-matter | | | |
| | | | abnormality in | | | |
| | | | basal ganglia | | | |
| | | | and cerebral | | | |
| | | | cortex | | | |
| | | | compatible | | | |
| | | | with delayed | | | |
| | | | myelination | | | |
| 12-F | history of | 17 y, | 19 y, mild | Negative for SCA 1-3, | [=] + [=] | [=] + [=] |
| | normal | behavioral | atrophy, slight | 6-8, DRPLA, FRDA, | | |
| | puberty | problems, | periventricular | FXTAS | | |
| | 24 y, onset of | followed by | and basal | Negative genetic | | |
| | hypogonado- | tremor, | ganglia T2 and | testing for MELAS, | | |
| | tropic | dysarthria, | FLAIR | MERRF, NARP, | | |
| | hypogonadism | and gait | hyperintensity | mitochondrial | | |
| | | disturbance | 21 y, increased | deletions | | |
| | | 19 y, bilateral | hyperintensity | | | |
| | | internuclear | in brainstem, | | | |
| | | ophthalmoplegia | thalami, | | | |
| | | 24 y, marked | internal | | | |
| | | emotional | capsules, | | | |
| | | lability and | insulae | | | |
| | | disinhibition | 23 y, increased | | | |
| | | 25 y, speech | cerebral | | | |
| | | impairment | volume loss, | | | |
| | | | no change in | | | |
| | | | T2 | | | |
| | | | hyperintensity | | | |
| | | | 24 y, little | | | |
| | | | change | | | |

AOA, ataxia with oculomotor apraxia;
AVED, ataxia with vitamin E deficiency;
DRPLA, dentatorubral-pallidoluysian atrophy;
FRDA, Friedreich's ataxia;
FXTAS, fragile X-associated tremor/ataxia syndrome;
HD, Huntington's disease;
HDL, Huntington's disease- like;
MELAS, mitochondrial myopathy, encephalopathy, lactic acidosis, and stroke-like episodes;
MERRF, myoclonic epilepsy associated with ragged red fibers;
MSS, Marinesco-Sjögren syndrome;
NARP, neuropathy, ataxia, and retinitis pigmentosa;
SANDO, sensory ataxic neuropathy, dysarthria, and ophthalmoparesis;
SBMA, spinobulbar muscular atrophy;
SCA, spinocerebellar ataxia.

### Example 2

### Cell Specific Cell Death Mechanisms

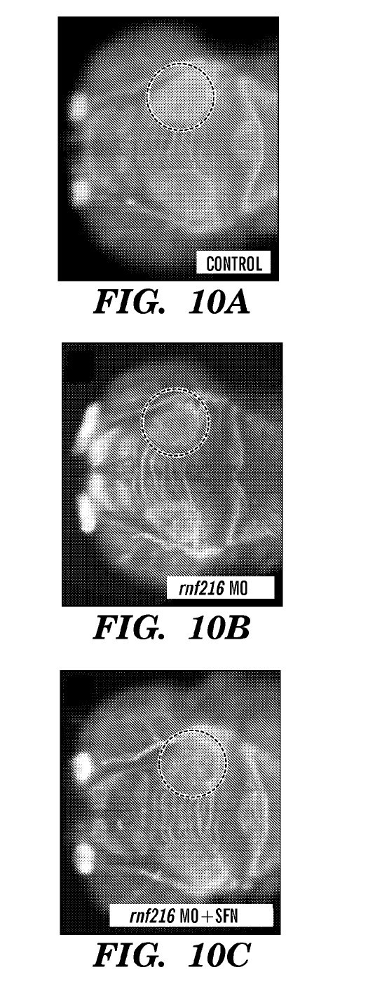

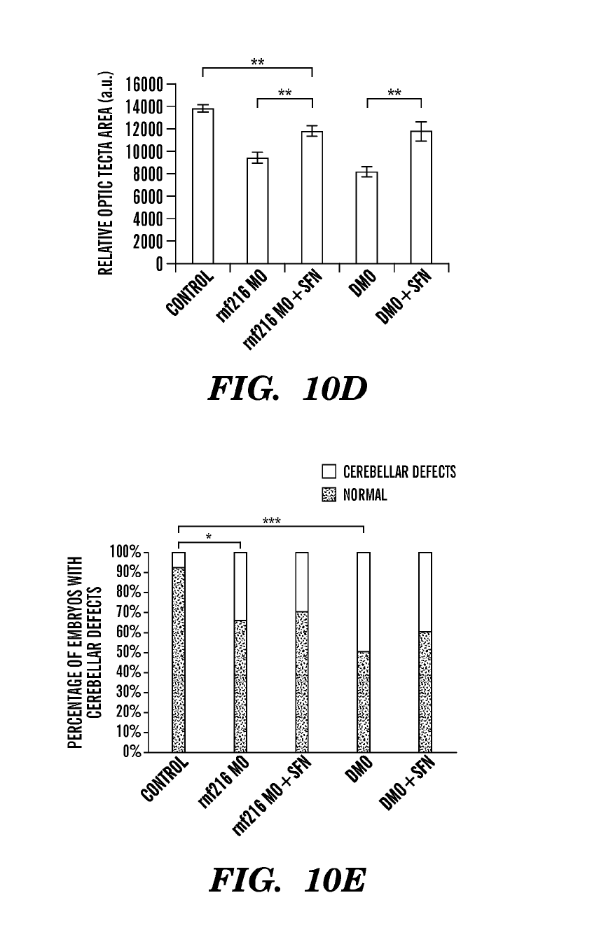

The genetics data to date indicates a role for the UPS and autophagy in GHS. As such, activation of UPS-mediated protein degradation through proteasomal agonists can be of clinical utility. SFN (1-isothio-cyanato-4(R)-methylsulfinylbutane, extracted from cruciferous vegetables) is a chemopreventive compound and has been utilized in animal studies and anti-cancer trials.^{1,2} Efficacy and potency profiles for SFN in an in vivo zebrafish assay of neuronal survival and degeneration were established. rnf216 and rnf216+otud4 (DMO) morphants were co-injected with SFN. At 3 days post fertilization (dpf) of the CNS, a significant reduction of both rnf216 and DMO morphants with optic tectum defects (from 32% to 15% for rnf216 and from 41% to 15% for DMO, respectively) with SFN injection was observed (p<0.001; FIGS. 10A-10D). However, co-injection of SFN with either rnf216 MO or DMO failed to ameliorate the cerebellar disorganization phenotype (FIGS. 10A-10E). These results suggest that upregulation of proteasomal activity can ameliorate the cortical degeneration phenotype but not the cerebellar disorganization. Thus, two independent mechanisms may regulate cortical vs. cerebellar neurotoxicity, with proteasomal activation being sufficient to rescue the cortical phenotype. This hypothesis will be tested in Rnf216^{-/-} and Otud4^{-/-} mice.

Unique neurologic & reproductive subphenotypes are present in patients with RNF216 mutations.

It is demonstrated herein that the neuropathology of GHS can be defined with the brain of a mutation+patient.

Stub1^{-/-} mice have cerebellar and hypogonadotropic phenotypes.

Upregulation of the proteasome in zebrafish rescues neuronal but not cerebellar phenotypes, pointing to a potential therapy. Whether the pathophysiology of the cerebellum is different from the hypothalamus/pituitary will be tested in Rnf216^{-/-} & Otud4^{-/-} mice.

1.-34. (canceled)

35. A method of treating a reproductive or sex hormone-dependent condition in a subject in need thereof, the method comprising administering to the subject a proteasome agonist selected from the group consisting of:
SFN (1-isothio-cyanato-4(R)-methylsulfinylbutane); (methylsulfonyl)cyclohexylmethylisothiocyanante; oleuropein; betulinic acid; and derivatives thereof.

36. The method of claim 35, wherein the reproductive disease is selected from the group consisting of:
hypogonadotropic hypogonadism; delayed puberty; amenorrhea; irregular menstrual function; polycystic ovary syndrome; erectile dysfunction; decreased libido; azoospermia; and infertility.

37. A method of treating a subject, the method comprising:
detecting, in a sample obtained from a subject, the presence of a deleterious mutation in RNF216 and optionally, OTUD4; and
administering a treatment for Gordon Holmes syndrome to the subject.

38. The method of claim 37, wherein the treatment increases the expression or activity of RNF216 and optionally, OTUD4.

39. The method of claim 37, wherein the treatment is selected from the group consisting of:
an ubiquitin acting drug; a drug acting within the proteasome; a drug acting within the ataxia protein protein interaction network; a USP14 inhibitor; IU1; calcium channel blockers; pioglitazone; conenzyme Q10; idebenone; overexpression of heat shock proteins; inhibitors of ATXN1; zolpidem; varenicline; implantation of syngenic cerebellar, gonadotroph, or hypothalaus cells; and implantation of genetically-modified cerebellar, gonadotroph, or hypothalaus cells.

40. The method of claim 39, wherein the drug acting within the proteasome is a proteasome agonist selected from the group consisting of:
SFN (1-isothio-cyanato-4(R)-methylsulfinylbutane); (methylsulfonyl)cyclohexylmethylisothiocyanante; oleuropein; betulinic acid; and derivatives thereof.

41. The method of claim 37, wherein the detecting further comprises measuring whether the subject is homozygous or heterozygous for the deleterious mutation.

42. The method of claim 37, wherein the deleterious mutation results in a truncation of the polypeptide as compared to SEQ ID NO: 1 or SEQ ID NO: 2.

43. The method of claim 37, wherein the detecting comprises detecting the size of the RNF216 and optionally, OTUD4 polypeptide.

44. The method of claim 37, wherein the deleterious mutation results in a polypeptide which differs by at least 10% from a polypeptide having the sequence of SEQ ID NO: 1 or SEQ ID NO: 2.

45. The method of claim 37, wherein the detecting comprises determining the amino acid sequence of the RNF216, and optionally, OTUD4 polypeptide.

46. The method of claim 37, wherein the detecting comprises determining the nucleic acid sequence of a nucleic acid molecule encoding RNF216 and optionally, OTUD4.

47. The method of claim 37, wherein the deleterious mutation of RNF216 is selected from the group consisting of:
c.2251C>T; p.R751C; c.615_{—}616delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; p.R717C; mutation of R751; and mutation of R717.

48. The method of claim 47, wherein the deleterious mutation of RNF216 is selected from the group consisting of:
c.2251C>T; p.R751C; c.615_{—}616delGA; p.E205DfsX15; a premature termination after amino acid 219; a frameshift mutation in amino acid 205; c.1791T>A; p.C597X; a premature termination in or after exon 11; c.414delG; p.G138GfsX74; c.721C>T; p.Q241X; a premature termination after amino acid 240; a premature termination after amino acid 219; c.2149C>T; and p.R717C.

49. The method of claim 37, wherein the deleterious mutation of OTUD4 is selected from the group consisting of:
998G>T and G333V.

50. A method of treating Gordon Holmes Syndrome in a subject in need thereof, the method comprising administering to the subject a proteasome agonist selected from the group consisting of:
SFN (1-isothio-cyanato-4(R)-methylsulfinylbutane); (methylsulfonyl)cyclohexylmethylisothiocyanante; oleuropein; betulinic acid; and derivatives thereof.
